(19) United States
(12) Patent Application Publication
(10) Pub. No.: US 2025/0270592 A1
(43) Pub. Date: Aug. 28, 2025

(54) REPROGRAMMING OF CELLS WITH SELF-AMPLIFYING RNA

(71) Applicant: STEMCELL TECHNOLOGIES CANADA INC., Vancouver (CA)

(72) Inventors: Chun-I WU, Watertown, MA (US); Devin BRIDGEN, Watertown, MA (US); Jonathan B. GILBERT, Watertown, MA (US)

(21) Appl. No.: 18/867,542

(22) PCT Filed: May 24, 2023
(86) PCT No.: PCT/US2023/023375

§ 371 Date: Nov. 20, 2024

Related U.S. Application Data
(60) Provisional application No. 63/345,339, filed on May 24, 2022.

(51) Int. Cl. C12N 15/89; C12N 5/0793

(52) U.S. Cl. CPC C12N 15/89; C12N 5/0619; C12N 2506/45; C12N 2820/00

(57) ABSTRACT

The present disclosure provides methods for reprogramming a cell, wherein the method comprises passing a cell suspension comprising the cell and a self-amplifying RNA encoding a reprogramming factor through a constriction, wherein the constriction deforms the cell, thereby causing a perturbation of the cell such that the reprogramming factor enters the cell.

### CROSS-REFERENCE TO RELATED APPLICATIONS

This PCT application claims the priority benefit of U.S. Provisional Application No. 63/345,339, filed May 24, 2022, which is herein incorporated by reference in its entirety.

### FIELD OF DISCLOSURE

The present disclosure relates generally to methods of reprogramming cells to differentiate into different types of cells (e.g., neurons) through the use of one or more constrictions and self-amplifying RNA encoding a reprogramming factor.

### BACKGROUND OF DISCLOSURE

Regenerative medicine based on cell replacement is on the cusp of major health impacts. However, significant challenges remain, particularly in manufacturing consistent, efficient, and cost-effective functional engraftable cells. While induced pluripotent stem cell (iPSC) differentiation and somatic cell transdifferentiation (i.e., direct reprogramming) are capable of producing various cells of interest, current methods are often cumbersome and inefficient. For instance, iPSC differentiation methods currently available can typically take up to several weeks and in some instances, even months to get to the desired terminal cell types. Furthermore, the resulting cell product is inevitably a heterogeneous population of cells with the desired cells present in wide range of frequency. Similarly, with somatic cell transdifferentiation, reprogramming by lentiviral vectors has been useful, but this method presents the risk of insertional mutagenesis. And, with methods such as electroporation and lipofection, they can negatively affect cell health and recovery of sensitive primary cells, as well as causing other cytotoxicity issues. Therefore, there remains a need for more efficient and cost-effective approaches to delivering differentiation or reprogramming factors to generate cells that can be used for various therapeutic applications, such as in regenerative medicine.

### SUMMARY OF DISCLOSURE

Provided herein is a method of inducing an expression of a reprogramming factor in a cell, comprising passing a cell suspension, which comprises the cell, through a constriction under a set of parameters, wherein passing the cell suspension through the constriction under the set of parameters causes a perturbation within the membrane of the cell, such that a self-amplifying RNA encoding the reprogramming factor can enter the cell through the perturbation when contacted with the cell, and thereby, resulting in the expression of the reprogramming factor in the cell.

In some aspects, the reprogramming factor is expressed in the cell for at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, or at least about 25 days after the self-amplifying RNA enters the cell through the perturbation. In some aspects, the expression of the reprogramming factor in the cell is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least 45-fold, or at least about 50-fold after the self-amplifying RNA enters the cell through the perturbation.

Also provided herein is a method of enhancing the expression of a reprogramming factor in a cell, comprising passing a cell suspension, which comprises the cell, through a constriction under a set of parameters, wherein passing the cell suspension through the constriction under the set of parameters causes a perturbation within the membrane of the cell, such that a self-amplifying RNA encoding the reprogramming factor can enter the cell through the perturbation when contacted with the cell, and thereby, resulting in enhanced expression of the reprogramming factor, as compared to a corresponding expression in a reference cell.

In some aspects, the reference cell comprises a corresponding cell that was passed through the constriction under the set of parameters, and wherein the reference cell was contacted with a non-self-amplifying RNA encoding the reprogramming factor. In some aspects, the reference cell comprises a corresponding cell that was not passed through the constriction under the set of parameters, and wherein the reference cell was contacted with the self-amplifying RNA encoding the reprogramming factor.

In some aspects, the enhanced expression comprises (i) an increased expression level, (ii) a longer duration of expression, or (iii) both (i) and (ii). In some aspects, the expression level of the reprogramming factor in the cell is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, at least about 75-fold, at least about 100-fold, at least about 150-fold, at least about 200-fold, at least about 250-fold, at least about 300-fold, at least about 350-fold, at least about 400-fold, at least about 450-fold, or at least about 500-fold, as compared to that of the reference cell. In some aspects, the duration of the expression of the reprogramming factor in the cell is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, at least about 75-fold, at least about 100-fold, at least about 150-fold, at least about 200-fold, at least about 250-fold, at least about 300-fold, at least about 350-fold, at least about 400-fold, at least about 450-fold, or at least about 500-fold, as compared to that of the reference cell.

Present disclosure further provides a method of method of reprogramming a cell, comprising passing a cell suspension, which comprises the cell, through a constriction under a set of parameters, wherein passing the cell suspension through the constriction under the set of parameters causes a perturbation within the membrane of the cell, such that a self-amplifying RNA encoding a reprogramming factor can enter the cell through the perturbation when contacted with the cell, and thereby, reprogramming the cell.

In some aspects, the reprogramming factor is expressed in the cell for at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, or at least about 25 days after the self-amplifying RNA enters the cell through the perturbation. In some aspects, the expression of the reprogramming factor in the cell is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least 45-fold, or at least about 50-fold after the self-amplifying RNA enters the cell through the perturbation.

Some aspects of the present disclosure provides a method of enhancing the reprogramming of a cell, comprising passing a cell suspension, which comprises the cell, through a constriction under a set of parameters, wherein passing the cell suspension through the constriction under the set of parameters causes a perturbation within the membrane of the cell, such that a self-amplifying RNA encoding a reprogramming factor can enter the cell through the perturbation when contacted with the cell, and thereby, resulting in enhanced reprogramming of the cell, as compared to a reference method.

In some aspects, the reference method comprises passing the cell suspension through the constriction under the set of parameters and contacting the cell suspension with a non-self-amplifying RNA encoding the reprogramming factor. In some aspects, the reference method does not comprise passing the cell suspension through the constriction under the set of parameters, and wherein the cell suspension is contacted with the self-amplifying RNA.

In some aspects, the enhanced reprogramming of the cell comprises (i) a greater number of cells having been reprogrammed, (ii) a shorter duration of time required to reprogram cells, or (iii) both (i) and (ii). In some aspects, the number of cells that have been reprogrammed is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, at least about 75-fold, at least about 100-fold, at least about 150-fold, at least about 200-fold, at least about 250-fold, at least about 300-fold, at least about 350-fold, at least about 400-fold, at least about 450-fold, or at least about 500-fold, as compared to the reference method. In some aspects, the duration of time required to reprogram the cells is decreased by at least about 5%, at least about 10%, at least about 20%, at least about 30%, at least about 40%, at least about 50%, at least about 60%, at least about 70%, at least about 80%, at least about 90%, or about 100%, as compared to the reference method.

In some aspects, the reprogramming of the cell comprises inducing the cell to differentiate into a neuron. In some aspects, the reprogramming of the cell comprises inducing the expression of other reprogramming factor in the cell.

Also provided herein is a method of producing a neuron, comprising passing a cell suspension through a constriction under a set of parameters, wherein passing the cell suspension through the constriction under the set of parameters causes a perturbation within the membrane of a cell which is present in the cell suspension, such that a self-amplifying RNA encoding a reprogramming factor can enter the cell through the perturbation when contacted with the cell, and thereby, resulting in the differentiation of the cell into a neuron. Provided herein is a method of enhancing the production of a neuron, comprising passing a cell suspension through a constriction under a set of parameters, wherein passing the cell suspension through the constriction under the set of parameters causes a perturbation within the membrane of a cell which is present in the cell suspension, such that a self-amplifying RNA encoding a reprogramming factor can enter the cell through the perturbation when contacted with the cell, and thereby, resulting in enhanced production of the neuron, as compared to a reference method.

In some aspects, the reference method comprises passing the cell suspension through the constriction under the set of parameters and contacting the cell suspension with a non-self-amplifying RNA encoding the reprogramming factor. In some aspects, the reference method does not comprise passing the cell suspension through the constriction under the set of parameters, and wherein the cell suspension is contacted with the self-amplifying RNA.

For any of the methods provided herein (e.g., such as those described above), in some aspects, the methods further comprise contacting the cell suspension with the self-amplifying RNA prior to the passing of the cell suspension through the constriction. In some aspects, the cell is in contact with the self-amplifying RNA prior to the passing of the cell suspension through the constriction. In some aspects, the methods further comprise contacting the cell suspension with the self-amplifying RNA during the passing of the cell suspension through the constriction. In some aspects, the cell is in contact with the self-amplifying RNA during the passing of the cell suspension through the constriction. In some aspects, the methods further comprise contacting the cell suspension with the self-amplifying RNA after the passing of the cell suspension through the constriction. In some aspects, the cell is in contact with the self-amplifying RNA after the passing of the cell suspension through the constriction.

For any of the methods provided herein (e.g., such as those described above), in some aspects, the reprogramming factor comprises a transcription factor. In some aspects, the reprogramming factor comprises a neurogenin-2 (Ngn2; Neurog2), Atonal BHLH Transcription Factor 1 (Atoh1), Achaete-Scute Family BHLH Transcription Factor 1 (Ascl1), Nuclear receptor subfamily 4 group A member 2 (NR4A2), LIM Homeobox Transcription Factor 1 Alpha (Lmx1a), Engrailed homobox 1 (EN1), POU Class 3 Homeobox 2 (POU3F2; Brn2), Myelin Transcription Factor 1 Like (Myt11), Forkhead Box A2 (Foxa2), Paired Like Homeodomain 3 (Pitx3), SRY-Box Transcription Factor 2 (SOX2), micro-RNA 124 (mir124), or combinations thereof.

For any of the methods provided herein (e.g., such as those described above), in some aspects, the methods further comprise contacting the cell suspension with a payload, such that the payload can also enter the cell through the perturbation when contacted with the cell. In some aspects, the method comprises contacting the cell suspension with the payload prior to the passing of the cell suspension through the constriction. In some aspects, the cell is in contact with the payload prior to the passing of the cell suspension through the constriction. In some aspects, the method comprises contacting the cell suspension with the payload during the passing of the cell suspension through the constriction. In some aspects, the cell is in contact with the payload during the passing of the cell suspension through the constriction. In some aspects, the methods comprise contacting the cell suspension with the payload after the passing of the cell suspension through the constriction. In some aspects, the cell is in contact with the payload after the passing of the cell suspension through the constriction.

Where the cell is contacted with both the payload and the saRNA, in some aspects, the self-amplifying RNA and the payload are contacted with the cell concurrently. In some aspects, the self-amplifying RNA and the payload are contacted with the cell sequentially.

In some aspects, the payload comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, the nucleic acid comprises a DNA, RNA, or both. In some aspects, the DNA comprises a recombinant DNA, a cDNA, a genomic DNA, or combinations thereof. In some aspects, the RNA comprises a siRNA, non-self-amplifying mRNA, microRNA (miRNA), IncRNA, tRNA, shRNA, self-amplifying mRNA (saRNA), or combinations thereof. In some aspects, the small molecule comprises an impermeable small molecule.

In some aspects, the payload comprises an additional reprogramming factor. In some aspects, the additional reprogramming factor comprises a neurogenin-2 (Ngn2; Neurog2), Atonal BHLH Transcription Factor 1 (Atoh1), Achaete-Scute Family BHLH Transcription Factor 1 (Ascl1), Nuclear receptor subfamily 4 group A member 2 (NR4A2) (, LIM Homeobox Transcription Factor 1 Alpha (Lmx1a), Engrailed homobox 1 (EN1), POU Class 3 Homeobox 2 (POU3F2; Brn2), Myelin Transcription Factor 1 Like (Myt1l), Forkhead Box A2 (Foxa2), Paired Like Homeodomain 3 (Pitx3), SRY-Box Transcription Factor 2 (SOX2), micro-RNA 124(mir124), or combinations thereof.

For any of the methods provided herein (e.g., such as those described above), in some aspects, the cell comprises stem cells, somatic cells, or both. In some aspects, the stem cells are induced pluripotent stem cells (iPSCs), embryonic stem cells, tissue-specific stem cells, mesenchymal stem cells, or combinations thereof. In some aspects, the stem cells are iPSCs. In some aspects, the somatic cells comprise blood cells. In some aspects, the blood cells are PBMCs. In some aspects, the PBMCs comprise immune cells. In some aspects, the immune cells comprise a T cell, B cell, natural killer (NK) cell, dendritic cell (DC), NKT cell, mast cell, monocyte, macrophage, basophil, eosinophil, neutrophil, DC2.4 dendritic cell, or combinations thereof.

For any of the methods provided herein (e.g., such as those described above), in some aspects, the set of parameters is selected from a cell density; pressure; length, width, and/or depth of the constriction; diameter of the constriction; diameter of the cells; temperature; entrance angle of the constriction; exit angle of the constriction; length, width, and/or width of an approach region; surface property of the constriction (e.g., roughness, chemical modification, hydrophilic, hydrophobic); operating flow speed; payload concentration; viscosity, osmolarity, salt concentration, serum content, and/or pH of the cell suspension; time in the constriction; shear rate in the constriction; type of payload, or combinations thereof. In some aspects, the cell density is at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more. In some aspects, the pressure is at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

For any of the methods provided herein (e.g., such as those described above), in some aspects, the constriction is contained within a microfluidic chip. In some aspects, the diameter of the constriction is about 20%, about 30%, about 40%, about 50%, about 60%, about 70%, about 80%, about 90%, or about 99% of the diameter of the one or more cells of the population of cells.

In some aspects, the length of the constriction is between about 0 μm to about 100 μm. In some aspects, the length of the constriction is less than about 1 μm. In some aspects, the length of the constriction is less than about 0.1 μm, less than about 0.2 μm, less than about 0.3 μm, less than about 0.4 μm, less than about 0.5 μm, less than about 0.6 μm, less than about 0.7 μm, less than about 0.8 μm, less than about 0.9 μm, less than about 1 μm, less than about 2.5 μm, less than about 5 μm, less than about 7.5 μm, less than about 10 μm, less than about 12.5 μm, less than about 15 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2.5 μm, about 5 μm, about 7.5 μm, about 10 μm, about 12.5 μm, about 15 μm, about 20 μm, about 30 μm, about 40 μm, about 50 μm, about 60 μm, about 70 μm, about 80 μm, about 90 μm, or about 100 μm. In some aspects, the length of the constriction is about 10 μm.

In some aspects, the width of the constriction is between about 0 μm to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width of the constriction is between about 3 μm to about 10 μm. In some aspects, the width of the constriction is about 3 μm, about 4 μm, about 5 μm, about 6 μm, about 7 μm, about 8 μm, about 9 μm, or about 10 μm. In some aspects, the width of the constriction is about 6 μm.

In some aspects, the depth of the constriction is at least about 1 μm. In some aspects, the depth of the constriction is at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth of the constriction is about 5 μm, about 10 μm, about 20 μm, about 30 μm, about 40 μm, about 50 μm, about 60 μm, about 70 μm, about 80 μm, or about 90 μm. In some aspects, the depth of the constriction is about 70 μm.

In some aspects, the length of the constriction is about 10 μm, the width of the constriction is about 6 μm, and the depth of the constriction is about 70 μm.

In some aspects, the methods provided herein comprise contacting the cell suspension with multiple self-amplifying RNAs, multiple payloads, or both (i) prior to the passing of the cell suspension through the constriction, (ii) during the passing of the cell suspension through the constriction, (iii) after the passing of the cell suspension through the constriction, or (iv) any combination of (i) to (iii). In some aspects, the cell is in contact with the multiple self-amplifying RNAs, and wherein at least two or more of the multiple self-amplifying RNAs can enter the cell through the perturbation. In some aspects, the cell is in contact with the multiple payloads, and wherein at least two or more of the multiple payloads can enter the cell through the perturbation. In some aspects, the multiple self-amplifying RNAs comprise at least at least about 2, at least about 3, at least about 4, at least about 5, at least about 6, at least about 7, at least about 8, at least about 9, or at least about 10 or more self-amplifying RNAs. In some aspects, each of the multiple self-amplifying RNAs is different. In some aspects, at least two of the multiple self-amplifying RNAs are the same.

In some aspects, the multiple payloads comprise at least at least about 2, at least about 3, at least about 4, at least about 5, at least about 6, at least about 7, at least about 8, at least about 9, or at least about 10 or more payloads. In some aspects, each of the multiple payloads is different. In some aspects, at least two of the multiple payloads are the same.

In some aspects, the multiple self-amplifying RNAs are contacted with the cell concurrently. In some aspects, the multiple self-amplifying RNAs are contacted with the cell sequentially. In some aspects, the multiple payloads are contacted with the cell concurrently. In some aspects, the multiple payloads are contacted with the cell sequentially. In some aspects, the multiple self-amplifying RNAs and the multiple payloads are contacted with the cell concurrently. In some aspects, the multiple self-amplifying RNAs and the multiple payloads are contacted with the cell sequentially.

For any of the methods provided herein (e.g., such as those described above), in some aspects, the methods comprise passing the cell suspension through a plurality of constrictions. In some aspects, the plurality of constrictions comprise at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, each constriction of the plurality of constrictions is the same. In some aspects, one or more of the constrictions of the plurality of constrictions are different. In some aspects, the one or more of the constrictions differ in their length, depth, width, or combinations thereof.

In some aspects, each constriction of the plurality of constrictions is associated with the same self-amplifying RNA. In some aspects, one or more of the plurality of constrictions is associated with a different self-amplifying RNA. In some aspects, the plurality of constrictions comprise a first constriction associated with a first self-amplifying RNA and a second constriction associated with a second amplifying RNA, wherein the cell suspension passes through the first constriction such that the first self-amplifying RNA can enter the cell, and then the cell suspension passes through the second constriction such that the second self-amplifying RNA can enter the cell. In some aspects, the cell suspension passes through the second constriction at least about 1 minute, at least about 30 minutes, at least about 1 hour, at least about 6 hours, at least about 12 hours, or at least about 1 day after the cell suspension passes through the first constriction.

For any of the methods provided herein (e.g., such as those described above), in some aspects, the method further comprises contacting the cell suspension with an additional compound. In some aspects, the additional compound comprises a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, the additional compound is a nucleic acid encoding an enzyme that confers resistance to an antibiotic. In some aspects, the contacting of the additional compound with the cell suspension occurs concurrently with the self-amplifying RNA. In some aspects, the contacting of the additional compound with the cell suspension occurs prior to or after the contacting of the cell suspension with the self-amplifying RNA.

In some aspects, the method further comprises collecting the cell suspension that passed through the constriction and treating the cell suspension with the antibiotic. In some aspects, the enzyme comprises puromycin-N-acetyltransferase and the antibiotic is puromycin. In some aspects, after the treatment with the antibiotic, the proportion of cells comprising the self-amplifying RNA present in the cell suspension is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold.

Some aspects of the present disclosure is directed to a population of cells comprising a reprogramming factor, wherein the population of cells were produced using any of the methods provided herein. Also provided herein is a population of reprogrammed cells produced using any of the methods of the present disclosure. The present disclosure further provides a population of neurons, which have been produced using any of the methods provided herein.

Provided herein is a composition comprising any of the population of cells, population of reprogrammed cells, and/or population of neurons described herein. In some aspects, such a composition further comprises a pharmaceutically acceptable excipient.

Also provided herein is a kit comprising any of the population of cells, population of reprogrammed cells, and/or population of neurons described herein, and instructions for use.

The present disclosure further provides a composition comprising a population of cells and a self-amplifying RNA encoding a reprogramming factor under a set of parameters resulting in deformation of one or more cells of the population of cells, wherein the one or more cells comprise a perturbation in the cell membrane sufficient to allow the self-amplifying RNA to enter the one or more cells. In some aspects, the composition further comprises a pharmaceutically acceptable excipient.

Provided herein is a cell comprising a perturbation in the cell membrane due to a set of parameters which deform the cell, thereby causing the perturbation in the cell membrane of the cell, such that a self-amplifying RNA encoding a reprogramming factor can enter the cell. Also provided herein is a cell comprising a reprogramming factor, wherein the reprogramming factor entered the cell through a perturbation in the cell membrane due to a set of parameters which deformed the cell, thereby causing the perturbation in the cell membrane of the cell, and allowing a self-amplifying RNA encoding the reprogramming factor to enter the cell through the perturbation. Provided herein is a composition comprising such cells. In some aspects, such a composition further comprises a pharmaceutically acceptable excipient.

Some aspects of the present disclosure provides a method of treating a disease or disorder in a subject in need thereof, comprising administering to the subject any of the compositions provided herein. In some aspects, the disease or disorder comprises a neurological disorder. In some aspects, the neurological disorder comprises a Parkinson's disease.

### BRIEF DESCRIPTION OF FIGURES

FIGS. 1A-1D show GFP expression kinetics in iPSCs after squeeze delivery of either GFP-encoding saRNA or GFP-encoding mRNA (i.e., non-self-amplifying). Specifically, the following groups of iPSCs are shown: (1) received a high dose (1.5 mg/mL) of GFP-encoding saRNA and subsequently treated with puromycin (inverted triangle); (2) received a low dose (0.3 mg/mL) of GFP-encoding saRNA and subsequently treated with puromycin (diamond); (3) received a high dose (1.5 mg/mL) of GFP-encoding saRNA alone (i.e., no puromycin treatment) (square); (4) received a low dose (0.3 mg/mL) of GFP-encoding saRNA alone (dark circle); and (5) modified iPSCs comprising GFP-encoding mRNA (i.e., non-self-amplifying) alone (triangle). iPSCs that were squeeze processed without any payload were used as control (“squeeze delivery only”; light circle). GFP expression was observed at days 1, 3, 9, 15, and 21 post-squeeze delivery using flow cytometry. FIG. 1A provides a schematic of the experimental design. FIG. 1B shows GFP expression as a percentage of total iPSCs. FIG. 1C shows the mean fluorescence intensity (MFI) of GFP expression on GFP⁺ iPSCs. FIG. 1D provides flow cytometry histogram plots comparing GFP expression among the different iPSCs.

FIG. 2 shows the expression of NeuroD1 (left graph), FoxA2 (middle graph), and NR4A2 (right graph) in iPSCs after squeeze delivery of one of the following: (1) Ascl1-encoding saRNA+five different mRNAs encoding FoxA2, Lmx1A, NR4A2, Pitx3, and EN1 (triangle); (2) Ascl1-encoding saRNA alone (square); and (3) GFP-encoding saRNA alone (green). Expression was measured at days 1 and 4 post-squeeze delivery and normalized to GFP-encoding saRNA fold increase.

FIG. 3 shows the expression of tyrosine hydroxylase (TH) (left graph), Lmx1A (middle graph), and Pitx3 (right graph) in iPSCs after squeeze delivery of one of the following: (1) Ascl1-encoding saRNA+five different mRNAs encoding FoxA2, Lmx1A, NR4A2, Pitx3, and EN1 (triangle); (2) Ascl1-encoding saRNA alone (square); and (3) GFP-encoding saRNA alone (green). Expression was measured at days 1 and 4 post-squeeze delivery and normalized to GFP-encoding saRNA fold increase.

FIG. 4 shows the expression of TUJ1 (early neuronal marker), tyrosine hydroxylase (TH) (dopamine neuronal marker), and MAP2 (mature neuronal marker) in iPSCs after squeeze delivery of Ascl1-encoding saRNA+five different mRNAs encoding FoxA2, Lmx1A, NR4A2, Pitx3, and EN1, as measured using immunofluorescence microscopy. In the first column, the green and red arrows represent those iPSCs with high and low TH expression, respectively. In the second column, the green arrows represent those iPSCs with high TH expression and further expressing TUJ1 (top box) or MAP2 (bottom box).

FIG. 5 shows the expression of NeuroD1 (left graph) and FoxA2 (right graph) in iPSCs after squeeze delivery of one of the following: (1) Ascl1-encoding saRNA+six different mRNAs encoding Ascl1, FoxA2, Lmx1a, NR4A2, Pitx3, and EN1 (inverted triangle); (2) Ascl1-encoding saRNA alone (diamond); (3) six different mRNAs encoding Ascl1, FoxA2, Lmx1a, NR4A2, Pitx3, and EN1+mRNA encoding puromycin N-acetyltransferase (PAC mRNA) (triangle); and (4) GFP-encoding saRNA alone (circle). Expression was measured at days 1 and 4 post-squeeze delivery and normalized to GFP-encoding saRNA fold increase.

FIG. 6 shows the expression of NR4A2 (1^{st} graph from the left), Pitx3 (2^{nd} graph), tyrosine hydroxylase (TH) (3^{rd} graph), and Lmx1a (4^{th} graph) in iPSCs after squeeze delivery of one of the following: (1) Ascl1-encoding saRNA+six different mRNAs encoding Ascl1, FoxA2,Lmx1a, NR4A2, Pitx3, and EN1 (inverted triangle); (2) Ascl1-encoding saRNA alone (diamond); (3) six different mRNAs encoding Ascl1, FoxA2, Lmx1a, NR4A2, Pitx3, and EN1+mRNA encoding puromycin N-acetyltransferase (PAC mRNA) (triangle); and (4) GFP-encoding saRNA alone (circle). Expression was measured at days 1 and 4 post-squeeze delivery and normalized to GFP-encoding saRNA fold increase.

### DETAILED DESCRIPTION OF DISCLOSURE

The present disclosure is generally directed to methods of producing neurons. More particularly, the methods provided herein comprise intracellularly delivering a self-amplifying RNA encoding a payload (e.g., reprogramming factor) to cells (e.g., iPSCs), such that the cells are induced to differentiate into neurons. In some aspects, intracellularly delivering the self-amplifying RNA to the cells comprises passing a cell suspension comprising a population of cells through a constriction under one or more parameters, such that the cells are transiently deformed, resulting in the perturbation of the cell membrane of the cells. Such a perturbation allows the self-amplifying RNA to enter the cell when contacted with the self-amplifying RNA.

As further described herein, the delivery methods of the present disclosure (squeeze delivery) have certain distinct properties that are not shared by other delivery methods known in the art. For example, in addition to the improved ability to deliver various types of payloads into a cell, the squeeze processing methods described herein exert minimal lasting effects on the cells. Compared to traditional delivery methods such as electroporation, the squeeze processing methods of the present disclosure preserve both the structural and functional integrity of the squeezed cells. Contrary to the delivery methods provided herein, electroporation can induce broad and lasting alterations in gene expression, which can lead to non-specific activation of cells (e.g., human T cells) and delayed proliferation upon antigen stimulation. With the present methods, any alterations to the cells (e.g., perturbations in the cell membrane) is transient and quickly repaired once the cells are removed from the constriction. Non-limiting examples of the various aspects are shown in the present disclosure.

### I. General Techniques

Some of the techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F.M. Ausubel, et al. eds., 2003); the series Methods in Enzymology (Academic Press, Inc.); PCR 2: A Practical Approach (M.J. MacPherson, B.D. Hames and G.R. Taylor eds., 1995); Antibodies, A Laboratory Manual (Harlow and Lane, eds., 1988); Culture of Animal Cells: A Manual of Basic Technique and Specialized Applications (R.I. Freshney, 6^{th} ed., J. Wiley and Sons, 2010); Oligonucleotide Synthesis (M.J. Gait, ed., 1984); Methods in Molecular Biology, Humana Press; Cell Biology: A Laboratory Notebook (J.E. Cellis, ed., Academic Press, 1998); Introduction to Cell and Tissue Culture (J.P. Mather and P.E. Roberts, Plenum Press, 1998); Cell and Tissue Culture: Laboratory Procedures (A. Doyle, J.B. Griffiths, and D.G. Newell, eds., J. Wiley and Sons, 1993-8); Handbook of Experimental Immunology (D.M. Weir and C.C. Blackwell, eds., 1996); Gene Transfer Vectors for Mammalian Cells (J.M. Miller and M.P. Calos, eds., 1987); PCR: The Polymerase Chain Reaction, (Mullis et al., eds., 1994); Current Protocols in Immunology (J.E. Coligan et al., eds., 1991); Short Protocols in Molecular Biology (Ausubel et al., eds., J. Wiley and Sons, 2002); Immunobiology (C.A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V.T. DeVita et al., eds., J.B. Lippincott Company, 2011).

### II. Definitions

For purposes of interpreting this specification, the following definitions will apply and whenever appropriate, terms used in the singular will also include the plural and vice versa. In the event that any definition set forth below conflicts with any document incorporated herein by reference, the definition set forth herein shall control. Additional definitions are set forth throughout the detailed description.

As used herein, the singular form term “a,” “an,” and “the” entity refers to one or more of that entity unless indicated otherwise. As such, the terms “a” (or “an” or “the”), “one or more,” and “at least one” can be used interchangeably herein.

It is understood that aspects and aspects of the disclosure described herein include “comprising,” “consisting,” and “consisting essentially of” aspects and aspects. It is also understood that wherever aspects and aspects are described herein with the language “comprising,” otherwise analogous aspects or aspects described in terms of “consisting of” and/or “consisting essentially of” are also provided.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

For all compositions described herein, and all methods using a composition described herein, the compositions can either comprise the listed components or steps, or can “consist essentially of” the listed components or steps. When a composition is described as “consisting essentially of” the listed components, the composition contains the components listed, and can further contain other components which do not substantially affect the methods disclosed, but do not contain any other components which substantially affect the methods disclosed other than those components expressly listed; or, if the composition does contain extra components other than those listed which substantially affect the methods disclosed, the composition does not contain a sufficient concentration or amount of the extra components to substantially affect the methods disclosed. When a method is described as “consisting essentially of” the listed steps, the method contains the steps listed, and can further contain other steps that do not substantially affect the methods disclosed, but the method does not contain any other steps which substantially affect the methods disclosed other than those steps expressly listed. As a non-limiting specific example, when a composition is described as “consisting essentially of” a component, the composition can additionally contain any amount of pharmaceutically acceptable carriers, vehicles, or diluents and other such components which do not substantially affect the methods disclosed.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “constriction” as used herein refers to a narrowed passageway. In some aspects, the constriction is a microfluidic channel, such as that contained within a microfluidic device. In some aspects, the constriction is a pore or contained within a pore. Where the constriction is a pore, in some aspects, the pore is contained in a surface. Unless indicated otherwise, the term constriction refers to both microfluidic channels and pores, as well as other suitable constrictions available in the art. Therefore, where applicable, disclosures relating to microfluidic channels can also apply to pores and/or other suitable constrictions available in the art. Similarly, where applicable, disclosures relating to pores can equally apply to microfluidic channels and/or other suitable constrictions available in the art.

The term “pore” as used herein refers to an opening, including without limitation, a hole, tear, cavity, aperture, break, gap, or perforation within a material. In some aspects, (where indicated) the term refers to a pore within a surface of a microfluidic device, such as those described in the present disclosure. In some aspects, (where indicated) a pore can refer to a pore in a cell wall and/or cell membrane.

The term “membrane” as used herein refers to a selective barrier or sheet containing pores. The term includes, but is not limited to, a pliable sheet-like structure that acts as a boundary or lining. In some aspects, the term refers to a surface or filter containing pores. This term is distinct from the term “cell membrane,” which refers to a semipermeable membrane surrounding the cytoplasm of cells.

The term “filter” as used herein refers to a porous article that allows selective passage through the pores. In some aspects, the term refers to a surface or membrane containing pores.

As used herein, the terms “deform” and “deformity” (including derivatives thereof) refer to a physical change in a cell. As described herein, as a cell passes through a constriction (such as those of the present disclosure), it experiences various forces due to the constraining physical environment, including but not limited to mechanical deforming forces and/or shear forces that causes perturbations in the cell membrane. As used herein, a “perturbation” within the cell membrane refers to any opening in the cell membrane that is not present under normal steady state conditions (e.g., no deformation force applied to the cells). Perturbation can comprise a hole, tear, cavity, aperture, pore, break, gap, perforation, or combinations thereof.

The term “heterogeneous” as used herein refers to something which is mixed or not uniform in structure or composition. In some aspects, the term refers to pores having varied sizes, shapes, or distributions within a given surface.

The term “homogeneous” as used herein refers to something which is consistent or uniform in structure or composition throughout. In some aspects, the term refers to pores having consistent sizes, shapes, or distribution within a given surface.

The term “polynucleotide” or “nucleic acid” as used herein refers to a polymeric form of nucleotides of any length, either ribonucleotides or deoxyribonucleotides. Thus, this term includes, but is not limited to, single-, double-or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as can typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. As described herein, a nucleic acid that can be delivered to a cell using the squeeze processing methods provided herein comprises a RNA (e.g., mRNA). As used herein, “RNA” comprises both self-amplifying RNA (e.g., self-amplifying mRNA) and non-self-amplifying RNA (e.g., non-self-amplifying mRNA). As used herein, the term “self-amplifying RNA” refers to a RNA molecule that can replicate in a host, resulting in an increase in the amount of RNA and proteins encoded by the RNA (e.g., reprogramming factors). As used herein, the term “mRNA” refers to any polynucleotides (either self-amplifying or non-self-amplifying) which encodes at least one polypeptide.

The terms “polypeptide” and “protein” are used interchangeably to refer to a polymer of amino acid residues, and are not limited to a minimum length. Such polymers of amino acid residues can contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications can be deliberate, as through site-directed mutagenesis, or can be accidental, such as through mutations of hosts which produce the proteins or errors due to PCR amplification.

### III. Methods of the Disclosure

In some aspects, the present disclosure relates to methods of delivering a self-amplifying RNA (saRNA) into a cell by passing the cells through a constriction (such as those described herein). As demonstrated herein, as the cells pass through the constriction, they become transiently deformed, such that cell membrane of the cells is perturbed. The perturbation within the cell membrane can allow various payloads (including self-amplifying RNA) to enter or loaded into the cell (e.g., through diffusion). The specific process by which the cells pass through a constriction and become transiently deformed is referred to herein as “squeeze processing,” “squeeze delivery,” or “squeezing.”

As demonstrated herein, through the methods provided herein, different aspects of a cell can be modified. In some aspects, by using a saRNA encoding a molecule that is capable of reprogramming a cell (i.e., “reprogramming factor”), the delivery methods provided herein can be used to produce different cells of interest. As further described herein, in some aspects, a reprogramming factor useful for the present disclosure comprises a differentiation factor. As used herein, the term “differentiation factor” refers to any agent that is capable of inducing the differentiation of a cell into a different type of cell. Unless indicated otherwise, the terms “reprogramming factor” and “differentiation factor” can be used interchangeably in the present disclosure. Accordingly, as used herein, the term “neuron reprogramming factor” or “neuron differentiation factor” can be used interchangeably and refer to an agent that is capable of reprogramming and/or inducing a cell to differentiate into a neuron. As used herein, a neuron reprogramming factor is not particularly limited, as long as the agent is capable of inducing a cell (e.g., stem cell or PBMCs) to differentiate into a neuron. Such neuron reprogramming factors are known in the art. Non-limiting examples of such factors include: neurogenin-2 (Ngn2; Neurog2), Atonal BHLH Transcription Factor 1 (Atoh1), Achaete-Scute Family BHLH Transcription Factor 1 (Ascl1), Nuclear receptor subfamily 4 group A member 2 (NR4A2) (also known as Nuclear receptor related 1 protein (Nurr1)), LIM Homeobox Transcription Factor 1 Alpha (Lmx1a), POU Class 3 Homeobox 2 (POU3F2; Brn2), Myelin Transcription Factor 1 Like (Myt11), Forkhead Box A2 (Foxa2), Paired Like Homeodomain 3 (Pitx3), Engrailed homobox 1 (EN1), SRY-Box Transcription Factor 2 (SOX2), micro-RNA 124 (mir124), or combinations thereof. In some aspects, the neuron reprogramming factor is Ngn2. In some aspects, the neuron reprogramming factor is Atoh1. In some aspects, the neuron reprogramming factor is Ascl1. In some aspects, the neuron reprogramming factor is NR4A2. In some aspects, the neuron reprogramming factor is Nurr1. In some aspects, the neuron reprogramming factor is Lmx1a. In some aspects, the neuron reprogramming factor is POU3F2. In some aspects, the neuron reprogramming factor is Myt11. In some aspects, the neuron reprogramming factor is Foxa2. In some aspects, the neuron reprogramming factor is Pitx3. In some aspects, the neuron reprogramming factor is SOX2.

While the above transcription factors generally activate signaling pathways that induce neuron differentiation, in some aspects, neuron reprogramming factors that are useful for the present disclosure can also inhibit signaling pathways, e.g., those pathways that interfere with neuron differentiation. Accordingly, in some aspects, a neuron reprogramming factor comprises a siRNA, such as that can inhibit p53 signaling. In some aspects, a neuron reprogramming factor comprises a miRNA (e.g., miR-214)

As further described and demonstrated herein, the above-described reprogramming factors can be delivered to a cell (e.g., stem cells or PBMCs) alone or in combination. For instance, such transcription factors can be encoded by a single saRNA (e.g., polycistronic saRNA), and the single saRNA can be delivered to a cell using squeeze delivery. In some aspects, multiple transcription factors can be delivered to a cell separately. For instance, as demonstrated herein, the delivery methods provided herein (e.g., squeeze processing) can be used to deliver a saRNA encoding a first transcription factor, a first mRNA encoding a transcription factor, a second mRNA encoding a transcription factor, a third mRNA encoding a transcription factor, a fourth mRNA encoding a transcription factor, and a fifth mRNA encoding a transcription factor. In some aspects, the delivery methods provided herein (e.g., squeeze processing) can be used to deliver a saRNA encoding a first transcription factor in combination with at least one mRNA, at least two mRNAs, at least three mRNAs, at least four mRNAs, at least five mRNAs, or at least six mRNAs, wherein each of the mRNAs encode a payload (e.g., reprogramming factor).

In some aspects, where combinations of reprogramming factors (or any other payloads described herein) are involved, they can be delivered to a cell using a single squeeze processing e.g., a cell suspension comprises the multiple payloads, which are delivered to the cell in combination; “concurrent delivery”). In some aspects, the multiple payloads (e.g., saRNA encoding a reprogramming factor in combination with one or more additional payloads) can be delivered to a cell sequentially. As used herein, the term “sequential delivery” refers to the delivery of multiple payloads to a cell, where a first payload (e.g., saRNA encoding a reprogramming factor) is delivered to the cell and then the second (or subsequent) payload (e.g., non-self-amplifying RNA encoding a reprogramming factor) is delivered to the cell. In some aspects, the first payload, the second payload, or both the first and second payloads can be delivered to the cell using squeeze processing. For instance, in some aspects, the first payload can be delivered to the cell using squeeze processing, and the second payload can be delivered to the cell using non-squeeze processing (e.g., transfection). In some aspects, the first payload can be delivered to the cell using non-squeeze processing (e.g., transfection), and the second payload can be delivered to the cell using squeeze processing. In some aspects, the first payload can be delivered to the cell using a first squeeze, and then the second payload can be delivered to the cell using a second squeeze (also referred to herein as “sequential squeeze” or “sequential squeeze processing”). Accordingly, sequential delivery useful for the present disclosure can comprise multiple squeeze processing. In some aspects, each of the multiple squeeze processing delivers a separate payload to the cell. In some aspects, one or more of the multiple squeeze processing do not involve the delivery of a payload. For instance, in some aspects, a sequential delivery method described herein comprises a first squeeze, a second squeeze, and a third squeeze, wherein the first squeeze comprises passing a cell without any payload through a first constriction, the second squeeze comprises passing the cell from the first squeeze through a second constriction to deliver a first payload (e.g., saRNA encoding a reprogramming factor) to the cell, and the third squeeze comprises passing the cell from the second squeeze through a third constriction to deliver a second payload to the cell. Not to be bound by any one theory, in some aspects, passing the cell through the first constriction without any payload (i.e., the first squeeze) can help prepare the cell for subsequent payload deliveries, e.g., can improve the delivery efficiency of the first payload and/or the second payload.

In some aspects, a combination of payloads can be delivered to a cell (e.g., stem cells or PBMCs) repeatedly. For instance, in some aspects, a combination of transcription factor is delivered to cells with a first squeeze processing; then, the combination of transcription factor is delivered to the cells again with a second squeeze processing. In some aspects, the first squeeze processing includes a microfluidic device (e.g., chip) with multiple rows of constrictions, such that the squeeze process occurs on a single microfluidic device (e.g., chip). As further described herein, in some aspects, the second squeeze processing can occur immediately after the cells have gone through the first squeeze processing (e.g., immediately after the cells pass through the constriction of the first squeeze processing). In some aspects, the second squeeze processing can occur after some time after the first squeeze processing (e.g., at least about 1 minute, at least about 30 minutes, at least about 1 hour, at least about 6 hours, at least about 12 hours, or at least about 1 day after the cells pass through the constriction of the first squeeze processing).

While the present disclosure generally discloses the use of saRNAs encoding reprogramming factors, it will be apparent to those skilled in the art that disclosures related to such saRNAs can equally apply to other types of payloads. Accordingly, as is apparent from the present disclosure, the squeeze processing or squeezing methods provided herein can also be used to deliver additional cargo (also referred to herein as “payload”) into a cell. Non-limiting examples of such payload are provided elsewhere in the present disclosure. Unless indicated otherwise, the terms “payload” and “reprogramming factor” are used interchangeably.

Accordingly, in some aspects, provided herein is a method of inducing an expression of a payload (e.g., reprogramming factor) in a cell, comprising passing a cell suspension, which comprises the cell, through a constriction under a set of parameters, wherein passing the cell suspension through the constriction under the set of parameters causes a perturbation within the membrane of the cell, such that a self-amplifying RNA encoding the payload can enter the cell through the perturbation when contacted with the cell, and thereby, resulting in the expression of the payload in the cell. As is apparent from the present disclosure, in some aspects, the payload is a reprogramming factor.

In some aspects, the above method allows for long-term expression of the encoded payload (e.g., reprogramming factor) when expressed in the cell. For instance, in some aspects, after squeeze delivery of a saRNA to the cell (e.g., iPSCs), the encoded payload is subsequently expressed in the cell for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1-year.

As demonstrated herein, Applicant has identified that the specific combination of squeeze delivery and saRNAs (also referred to herein as “delivery method provided herein” or variant thereof) is much more effective in inducing the expression of a payload (e.g., reprogramming factor) in a cell, as compared to other approaches in the art. Accordingly, in some aspects, provided herein is a method of enhancing the expression of a payload (e.g., reprogramming factor) in a cell, comprising passing a cell suspension, which comprises the cell, through a constriction under a set of parameters, wherein passing the cell suspension through the constriction under the set of parameters causes a perturbation within the membrane of the cell, such that a self-amplifying RNA encoding the payload can enter the cell through the perturbation when contacted with the cell, and thereby, resulting in enhanced expression of the payload, as compared to a corresponding expression in a reference cell. As used herein, the term “reference cell” refers to any cell (e.g., iPSCs) which has not been modified to comprise a saRNA using squeeze delivery. For instance, in some aspects, the reference cell comprises a corresponding cell that was passed through the constriction under the set of parameters but was not contacted with a saRNA. In some aspects, such a reference cell is contacted with a non-self-amplifying RNA (e.g., mRNA) encoding the payload. Accordingly, in some aspects, a reference cell comprises a corresponding cell (e.g., iPSCs) which has undergone squeeze processing but contacted with a mRNA encoding the payload. In some aspects, the reference cell comprises a corresponding cell that was contacted with the self-amplifying RNA encoding the payload, wherein the reference cell was not squeeze processed. For example, in some aspects, the reference cell is contacted with the saRNA using other delivery methods in the art (e.g., electroporation or lipofection).

In some aspects, compared to a reference cell (e.g., squeeze processed and contacted with a non-self-amplifying mRNA), the expression of the encoded payload is increased (e.g., greater expression level). In some aspects, compared to a reference cell (e.g., squeeze processed and contacted with a non-self-amplifying mRNA), the encoded payload is expressed in the cell for a much longer duration. In some aspects, compared to a reference cell (e.g., squeeze processed and contacted with a non-self-amplifying mRNA), both the expression of the encoded payload and the duration of expression is increased.

In some aspects, the expression level of the encoding payload (e.g., reprogramming factor) in the cell is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, at least about 75-fold, at least about 100-fold, at least about 150-fold, at least about 200-fold, at least about 250-fold, at least about 300-fold, at least about 350-fold, at least about 400-fold, at least about 450-fold, or at least about 500-fold, as compared to that of the reference cell. In some aspects, the duration of the expression of the encoded payload in the cell is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, at least about 75-fold, at least about 100-fold, at least about 150-fold, at least about 200-fold, at least about 250-fold, at least about 300-fold, at least about 350-fold, at least about 400-fold, at least about 450-fold, or at least about 500-fold, as compared to that of the reference cell.

As is apparent from the present disclosure, the methods provided herein can be particularly effective in delivering a reprogramming factor to a cell (e.g., iPSCs), and thereby, induce the reprogramming of a cell. Accordingly, in some aspects, the present disclosure is related to a method of reprogramming a cell, comprising passing a cell suspension, which comprises the cell, through a constriction under a set of parameters, wherein passing the cell suspension through the constriction under the set of parameters causes a perturbation within the membrane of the cell, such that a self-amplifying RNA encoding a reprogramming factor can enter the cell through the perturbation when contacted with the cell, and thereby, reprogram the cell. In some aspects, compared to other delivery methods in the art, the methods provided herein allow for much enhanced reprogramming of the cells. Therefore, in some aspects, provided herein is a method of enhancing the reprogramming of a cell, comprising passing a cell suspension, which comprises the cell, through a constriction under a set of parameters, wherein passing the cell suspension through the constriction under the set of parameters causes a perturbation within the membrane of the cell, such that a self-amplifying RNA encoding a reprogramming factor can enter the cell through the perturbation when contacted with the cell, and thereby, resulting in enhanced reprogramming of the cell, as compared to a reference method.

As used herein, the term “reference method” refers to a delivery method which does not comprise using squeeze processing to deliver a saRNA (e.g., encoding a reprogramming factor) to a cell (e.g., iPSCs). For instance, in some aspects, the reference method comprises passing the cell suspension through the constriction under the set of parameters, but the cells are not contacted with a saRNA described herein. In some aspects, such a method comprises contacting the cells with a non-self-amplifying RNA (e.g., mRNA) encoding the payload (e.g., reprogramming factor). Accordingly, in some aspects, a reference method comprises passing the cell suspension through the constriction under the set of parameters and contacting the cell suspension with a non-self-amplifying RNA encoding the reprogramming factor. In some aspects, a reference method comprises contacting a cell (e.g., iPSCs) with the saRNA but wherein the cell has not undergone squeeze processing. For example, in some aspects, the reference method comprises contacting the cell with the saRNA using other delivery methods in the art (e.g., electroporation or lipofection). In some aspects, compared to a reference method, the delivery methods provided

herein (e.g., squeeze processing in combination with saRNA) result in much greater number of reprogrammed cells. In some aspects, compared to a reference method, the delivery methods provided herein require much shorter duration of time to reprogram the cells. In some aspects, compared to a reference method, the delivery methods provided herein allow for both greater number of reprogrammed cells and shorter duration of time to reprogram the cells.

In some aspects, by using squeeze processing to deliver a saRNA encoding a reprogramming factor to a cell, the number of cells that have been reprogrammed is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, at least about 75-fold, at least about 100-fold, at least about 150-fold, at least about 200-fold, at least about 250-fold, at least about 300-fold, at least about 350-fold, at least about 400-fold, at least about 450-fold, or at least about 500-fold, as compared to the reference method. In some aspects, by using squeeze processing to deliver a saRNA encoding a reprogramming factor to a cell, the duration of time required to reprogram the cells is decreased by at least about 5%, at least about 10%, at least about 20%, at least about 30%, at least about 40%, at least about 50%, at least about 60%, at least about 70%, at least about 80%, at least about 90%, or about 100%, as compared to the reference method.

As is apparent from the present disclosure, the methods provided herein can be useful in producing many different types of cells. In some aspects, as demonstrated herein, the reprogramming factors described herein can induce the differentiation of cells (e.g., iPSCs) into neurons. Accordingly, in some aspects, provided herein is a method of producing a neuron, comprising passing a cell suspension through a constriction under a set of parameters, wherein passing the cell suspension through the constriction under the set of parameters causes a perturbation within the membrane of a cell which is present in the cell suspension, such that a self-amplifying RNA encoding a reprogramming factor can enter the cell through the perturbation when contacted with the cell, and thereby, resulting in the differentiation of the cell into a neuron. Non-limiting

Also provided herein is a method of enhancing the production of a neuron, comprising passing a cell suspension through a constriction under a set of parameters, wherein passing the cell suspension through the constriction under the set of parameters causes a perturbation within the membrane of a cell which is present in the cell suspension, such that a self-amplifying RNA encoding a reprogramming factor can enter the cell through the perturbation when contacted with the cell, and thereby, resulting in enhanced production of the neuron, as compared to a reference method.

In some aspects, using the methods provided herein (e.g., squeeze processing with saRNA encoding reprogramming factors), the number of neurons that is produced can be increased, e.g., compared to the reference method. In some aspects, with the methods provided herein, the duration of time required to produce neurons can be decreased, e.g., compared to the reference method. In some aspects, with the methods provided herein, both the number of neurons that can be produced and the duration of time required to produce the neurons can be decreased.

In some aspects, using the methods provided herein (e.g., squeeze processing with saRNA encoding reprogramming factors), the number of neurons that is produced is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, at least about 75-fold, at least about 100-fold, at least about 150-fold, at least about 200-fold, at least about 250-fold, at least about 300-fold, at least about 350-fold, at least about 400-fold, at least about 450-fold, or at least about 500-fold, as compared to the reference method. In some aspects, using the methods provided herein (e.g., squeeze processing with saRNA encoding reprogramming factors), the duration of time required to produce the neurons is decreased by at least about 5%, at least about 10%, at least about 20%, at least about 30%, at least about 40%, at least about 50%, at least about 60%, at least about 70%, at least about 80%, at least about 90%, or about 100%, as compared to the reference method.

The production of neurons (or any other cells of interest) can be assessed using any suitable methods known in the art. For instance, in some aspects, whether a cell (e.g., iPSCs) has been induced to differentiate into a neuron can be assessed by detecting the expression of certain markers associated with neurons (“neuronal marker”). Non-limiting examples of such markers include NeuroD1, FoxA2, NR4A2, tyrosine hydroxylase (TH), Lmx1a, Pitx3, Tuj1, Map2, or a combination thereof. In some aspects, a marker comprises a general neuronal marker (e.g., NeuroD1). In some aspects, a marker comprises a floor plate progenitor marker (e.g., FoxA2). In some aspects, a marker comprises a dopamine lineage marker (e.g., NR4A2, Pitx3, Lmx1a, and tyrosine hydroxylase). In some aspects, a marker comprises an early neuron marker (e.g., Tuj1). In some aspects, a marker comprises a mature neuron marker (e.g., Map2). Accordingly, unless indicated otherwise, the term “neuron,” as used herein, comprises any cell that expresses one or more of the above described neuronal markers.

Accordingly, in some aspects, with the delivery methods provided herein (e.g., using squeeze processing to deliver a saRNA encoding a reprogramming factor that is capable of inducing cells to differentiate into a neuron), the expression of any of the markers described above can be increased in the cell. In some aspects, compared to a reference method, the expression of a neuronal marker is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, at least about 50-fold, at least about 75-fold, at least about 100-fold, at least about 150-fold, at least about 200-fold, at least about 250-fold, at least about 300-fold, at least about 350-fold, at least about 400-fold, at least about 450-fold, or at least about 500-fold.

As is apparent from the present disclosure, for any of the delivery methods provided herein (e.g., using squeeze delivery to deliver a saRNA to a cell), in some aspects, the methods further comprise contacting the cells (e.g., which are to be reprogrammed) with the saRNA encoding a payload (e.g., reprogramming factor) prior to passing the cell suspension through the constriction. For instance, in some aspects, prior to passing the cell suspension through the constriction, the method comprises contacting the cell with a saRNA described herein (e.g., encoding a reprogramming factor) to produce the cell suspension (e.g., the cell suspension comprises both the cell to be reprogrammed and the reprograming factor prior to passing the cell suspension through the constriction). In some aspects, any of the delivery methods provided herein (e.g., using squeeze delivery to deliver a saRNA to a cell) further comprise contacting the cell with the saRNA encoding a payload as the cell suspension (comprising the cell) passes through the constriction. In some aspects, the cells are first contacted with the saRNA described herein during the passing of the cell suspension through the constriction. In some aspects, the cells are in contact with the saRNA both prior to the passing step (i.e., passing of the cell suspension through the constriction) and during the passing step. In some aspects, the method further comprises contacting the cells with the saRNA encoding a payload (e.g., reprogramming factor) after the passing of the cell suspension through the constriction. In some aspects, the cells are first contacted with the saRNA described herein after the passing of the cell suspension through the constriction. In some aspects, the cells are in contact with the saRNA prior to, during, and/or, after the passing step. As further described elsewhere in the present disclosure, when the cells are contacted with the saRNA encoding a payload (e.g., reprogramming factor) after the passing step, the contacting occurs soon after the cells have passed through the constriction, such that there are still perturbations within the cell membrane.

As used herein, “contacting” between a cell and a saRNA encoding a payload (e.g., reprogramming factor) does not require that the cell and the saRNA be in physical contact. As is apparent from the present disclosure, contacting between a cell and a saRNA occurs as long as the saRNA is capable of entering the cell once there are perturbations within the cell membrane of the cell. To help illustrate, in some aspects, a cell and a saRNA are in contact if they are both present within the same cell suspension, regardless of whether the cell and the saRNA are in physical contact or not.

### III.A. Cell Suspensions

In some aspects, a cell suspension described herein comprises any suitable cells known in the art that can be modified (e.g., by introducing a saRNA encoding a reprogramming factor using the squeeze processing methods provided herein.

In some aspects, the cells are stem cells. As used herein, the term “stem cells” refer to cells having not only self-replication ability but also the ability to differentiate into other types of cells (e.g., neurons). In some aspects, stem cells useful for the present disclosure comprise induced pluripotent stem cells (iPSCs), embryonic stem cells (ESCs), tissue-specific stem cells (e.g., liver stem cells, cardiac stem cells, or neural stem cells), mesenchymal stem cells, hematopoietic stem cells (HSCs), or combinations thereof. In some aspects, the stem cells are iPSCs.

In some aspects, the cells are somatic cells. As used herein, the term “somatic cells” refer to any cell in the body that are not gametes (sperm or egg), germ cells (cells that go on to become gametes), or stem cells. Non-limiting examples of somatic cells include blood cells, bone cells, muscle cells, nerve cells, or combinations thereof. In some aspects, somatic cells useful for the present disclosure comprise blood cells. In some aspects, the blood cells are peripheral blood mononuclear cells (PBMCs). As used herein, “PBMCs” refer to any peripheral blood cells having a round nucleus. In some aspects, PBMCs comprise an immune cell. As used herein the term “immune cell” refers to any cell that plays a role in immune function. In some aspects, immune cell comprises a T cell, B cell, natural killer (NK) cell, dendritic cell (DC), NKT cell, mast cell, monocyte, macrophage, basophil, eosinophil, neutrophil, DC2.4 dendritic cell, or combinations thereof. In some aspects, the blood cells are red blood cells. In some aspects, the cell is a cancer cell. In some aspects, the cancer cell is a cancer cell line cell, such as a HeLa cell. In some aspects, the cancer cell is a tumor cell. In some aspects, the cancer cell is a circulating tumor cell (CTC). In some aspects, the cell is a fibroblast cell, such as a primary fibroblast or newborn human foreskin fibroblast (Nuff cell). In some aspects, the cell is an immortalized cell line cell, such as a HEK293 cell or a CHO cell. In some aspects, the cell is a skin cell. In some aspects, the cell is a reproductive cell such as an oocyte, ovum, or zygote. In some aspects, the cell is a cluster of cells, such as an embryo, given that the cluster of cells is not disrupted when passing through the pore.

In some aspects, the cell suspension useful for the present disclosure comprises a mixed or purified population of cells. In some aspects, the cell suspension is a mixed cell population, such as whole blood, lymph, PBMCs, or combinations thereof. In some aspects, the cell suspension is a purified cell population. In some aspects, the cell is a primary cell or a cell line cell.

As demonstrated herein, the delivery of a saRNA encoding a payload (e.g., reprogramming factor) into a cell can be regulated through one or more parameters of the process in which a cell suspension is passed through a constriction. In some aspects, the specific characteristics of the cell suspension can impact the delivery of the saRNA into a cell. Such characteristics include, but are not limited to, osmolarity, salt concentration, serum content, cell concentration, pH, temperature or combinations thereof. Additional disclosure relating to such parameters are provided throughout the present disclosure.

In some aspects, the cell suspension comprises a homogeneous population of cells. In some aspects, the cell suspension comprises a heterogeneous population of cells (e.g., whole blood or a mixture of cells in a physiological saline solution or physiological medium other than blood). In some aspects, the cell suspension comprises an aqueous solution. In some aspects, the aqueous solution comprises a cell culture medium, PBS, salts, sugars, growth factors, animal derived products, bulking materials, surfactants, lubricants, vitamins, polypeptides, an agent that impacts actin polymerization, or combinations thereof. In some aspects, the cell culture medium comprises DMEM, OptiMEM, EVIDM, RPMI, or combinations thereof. Additionally, solution buffer can include one or more lubricants (pluronics or other surfactants) that can be designed to reduce or eliminate clogging of the surface and improve cell viability. Exemplary surfactants include, without limitation, poloxamer, polysorbates, sugars such as mannitol, animal derived serum, and albumin protein.

When the suspension includes certain types of cells, in some aspects, the cells can be treated with a solution that aids in the delivery of the payload (e.g., reprogramming factor) to the interior of the cell. In some aspects, the solution comprises an agent that impacts actin polymerization. In some aspects, the agent that impacts actin polymerization comprises Latrunculin A, Cytochalasin, Colchicine, or combinations thereof. For example, in some aspects, the cells can be incubated in a depolymerization solution, such as Lantrunculin A, for about 1 hour prior to passing the cells through a constriction to depolymerize the actin cytoskeleton. In some aspects, the cells can be incubated in Colchicine (Sigma) for about 2 hours prior to passing the cells through a constriction to depolymerize the microtubule network.

In some aspects, a characteristic of a cell suspension that can affect the delivery of a saRNA encoding a payload (e.g., reprogramming factor) into a cell is the viscosity of the cell suspension. As used herein, the term “viscosity” refers to the internal resistance to flow exhibited by a fluid. In some aspects, the viscosity of the cell suspension is between about 8.9×10⁻⁴ Pa·s to about 4.0×10⁻³ Pa·s, between about 8.9×10⁻⁴ Pa·s to about 3.0×10⁻³ Pa·s, between about 8.9×10⁻⁴ Pa·s to about 2.0×10⁻³ Pa·s, or between about 8.9×10⁻⁴ Pa·s to about 1.0×10⁻³ Pa·s. In some aspects, the viscosity is between about 0.89 cP to about 4.0 cP, between about 0.89 cP to about 3.0 cP, between about 0.89 cP to about 2.0 cP, or between about 0.89 cP to about 1.0 cP. In some aspects, a shear thinning effect is observed, in which the viscosity of the cell suspension decreases under conditions of shear strain. Viscosity can be measured by any suitable method known in the art, including without limitation, viscometers, such as a glass capillary viscometer or rheometers. A viscometer measures viscosity under one flow condition, while a rheometer is used to measure viscosities which vary with flow conditions. In some aspects, the viscosity is measured for a shear thinning solution such as blood. In some aspects, the viscosity is measured between about 0° C. and about 45° C. For example, the viscosity of the cell suspension can be measured at room temperature (e.g., about 20° C.), physiological temperature (e.g., about 37° C.), higher than physiological temperature (e.g., greater than about 37° C. to about 45° C. or more), reduced temperature (e.g., about 0°° C. to about 4° C.), or temperatures between these exemplary temperatures.

### III.B. Payloads

As described and demonstrated herein, the delivery methods provided in the present disclosure are particularly useful in intracellularly delivering a saRNA encoding a reprogramming factor, such as that which can induce the differentiation of a cell into a neuron. In some aspects, the delivery methods provided herein can further comprise contacting the cells with one or more additional payloads, such that multiple payloads can enter the cells through the perturbation when contacted with the cells. In some aspects, the additional payloads can be delivered to the cells concurrently with the saRNA described herein (e.g., encoding a reprogramming factor). For instance, in some aspects, the additional payload can be present in the cell suspension with a saRNA described herein prior to, during, and/or after the passing step, in which the cell suspension is passed through the constriction. In some aspects, the cell suspension comprises at least about 2, at least about 3, at least about 4, at least about 5, at least about 6, at least about 7, at least about 8, at least about 9, or at least about 10 or more additional payloads. Where multiple payloads are involved, in some aspects, the multiple payloads can be delivered to a cell as a single unit (e.g., a single saRNA encoding multiple payloads). In some aspects, the multiple payloads can be delivered to a cell as multiple units. For instance, the delivery methods provided herein can be used to deliver a saRNA encoding a first payload (e.g., reprogramming factor) and a non-self-amplifying RNA encoding a second payload (e.g., reprogramming factor).

As further described elsewhere in the present disclosure, in some aspects, a cell suspension can be passed through multiple constrictions. In such aspects, a payload can be loaded into a cell when the cells pass through one or more of the multiple constrictions. In some aspects, a payload is loaded into a cell each time the cells pass through one or more of the multiple constrictions. When multiple payloads are involved, each of the payloads can be the same. In some aspects, one or more of the payloads are different. Additional disclosure relating to the delivery of a saRNA described herein in combination with one or more additional payloads (also referred to herein as “multiplex delivery”) are provided elsewhere in the present disclosure.

As is apparent from the present disclosure, any suitable payloads known in the art can be delivered to a cell using the methods described herein (e.g., in combination with a saRNA described herein). Non-limiting examples of such payloads include a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, a small molecule comprises an impermeable small molecule. As used herein, an “impermeable small molecule” refers to a small molecule that naturally does not cross the cell membrane of a cell. In some aspects, an additional payload that can be delivered to a cell using the delivery methods provided herein comprises a nucleic acid. In some aspects, the nucleic acid comprises a DNA, RNA, or both. In some aspects, DNA comprises a recombinant DNA, a cDNA, a genomic DNA, or combinations thereof. In some aspects, RNA comprises a siRNA, a mRNA, a miRNA, a IncRNA, a tRNA, a shRNA, a self-amplifying mRNA (saRNA), or combinations thereof. In some aspects, the RNA is mRNA. In some aspects, the RNA is siRNA. In some aspects, the RNA is shRNA. In some aspects, the RNA is miRNA. In some aspects, the RNA is a saRNA. For instance, in some aspects, a delivery method described herein can comprise delivering a first saRNA encoding a first payload (e.g., reprogramming factor) and a second saRNA encoding a second payload (e.g., reprogramming factor) to a cell (e.g., iPSCs), wherein the first and second saRNAs are both delivered to the cell using squeeze processing. As further described herein, in some aspects, the first and second saRNAs can be delivered to the cell concurrently. In some aspects, the first and second saRNAs can be delivered to the cell sequentially. In some aspects, a delivery method provided herein comprises delivering a first saRNA encoding a first payload (e.g., reprogramming factor) and a second payload, which is a non-self-amplifying RNA encoding a second payload (e.g., reprogramming factor).

In some aspects, the additional compound is a nucleic acid encoding an enzyme that confers resistance to an antibiotic. Not to be bound by any one theory, by adding these compounds to the cell suspension comprising the plurality of cells and the reprogramming factor, it is possible to increase the purity of a mixture comprising the modified cells (i.e., reprogrammed/differentiated cells). By adding the particular antibiotic associated with the additional compound, the purity of a mixture comprising the modified cells (e.g., comprising differentiated neurons) can be increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold. Non-limiting example such an additional compound includes puromycin-N-acetyltransferase and the antibiotic is puromycin.

### III. C. Constrictions

### III.C.1. Microfluidic Channels

As described herein, a constriction is used to cause a physical deformity in the cells, such that perturbations are created within the cell membrane of the cells, allowing for the delivery of a payload (e.g., reprogramming factor) into the cell. In some aspects, a constriction is within a channel contained within a microfluidic device (referred to herein as “microfluidic channel” or “channel”). Where multiple channels are involved, in some aspects, the multiple channels can be placed in parallel and/or in series within the microfluidic device. In some aspects, the cells described herein can be passed through at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, the cells described herein are passed through more than about 1,000 separate constrictions. In some aspects, the multiple constrictions can be part of a single microfluidic device (e.g., multi-row constriction chip). In some aspects, one or more of the multiple constrictions can be part of different microfluidic devices. For instance, in some aspects, the cells described herein (e.g., stem cells or PBMCs) undergo a first squeeze processing, in which the cells pass through a first constriction in a first microfluidic device (e.g., chip). Then, after the cells have undergone the first squeeze processing (e.g., passed through the first constriction), the cells undergo a second squeeze processing, in which the cells pass through a second constriction in a second microfluidic device (e.g., chip). In some aspects, each of the constrictions are the same (e.g., has the same length, width, and/or depth). In some aspects, one or more of the constrictions are different. Where plurality of constrictions are used, the plurality of constrictions can comprise a first constriction which is associated with a first reprogramming factor, and a second constriction which is associated with a second reprogramming factor, wherein the cell suspension passes through the first constriction such that the first reprogramming factor is delivered to one or more cells of the plurality of cells, and then the cell suspension passes through the second constriction such that the second reprogramming factor is delivered to the one or more cells of the plurality of cells. In some aspects, the cell suspension is passed through the second constriction at least about 1 minute, at least about 30 minutes, at least about 1 hour, at least about 6 hours, at least about 12 hours, or at least about 1 day after the cell suspension is passed through the first constriction.

In some aspects, where the cell suspension is passed through multiple constrictions (e.g., multiple squeeze processing), the cells remain viable after passing through each constriction. As is apparent from the present disclosure, in some aspects, multiple constrictions can comprise two or more constrictions present within a single microfluidic device (e.g., multi-row constriction chip), such the cells pass through the multiple constrictions sequentially. In some aspects, the multiple constrictions are part of separate microfluidic devices, such that a first constriction is associated with a first microfluidic device and a second constriction is associated with a second microfluidic device. For instance, as demonstrated herein (e.g., Example 11), in some aspects, cells are passed through a first constriction (i.e., first squeeze processing), which is associated with a first microfluidic device (e.g., chip). After the cells have passed through the first constriction, the cells are passed through a second constriction (i.e., second squeeze processing), which is associated with a second microfluidic device (e.g., chip). In some aspects, after passing through the first constriction, the cells are cultured in a medium prior to passing the cells through the second constriction. In some aspects, the cells are cultured for at least about 1 minute, at least about 30 minutes, at least about 1 hour, at least about 6 hours, at least about 12 hours, or at least about 1 day before passing the cells through the second constriction. As is apparent from the present disclosure, in some aspects, the first and second constrictions have the same length, depth, and/or width. In some aspects, the first and second constrictions can have different length, depth, and/or width.

In some aspects, after passing through a constriction, at least about 50%, at least about 55%, at least about 60%, at least about 65%, at least about 70%, at least about 75%, at least about 80%, at least about 85%, at least about 90%, at least about 95%, or about 100% of the cells remain viable. Where the cells pass through multiple constrictions (e.g., part of a single microfluidic device or separate microfluidic devices), at least about 50%, at least about 55%, at least about 60%, at least about 65%, at least about 70%, at least about 75%, at least about 80%, at least about 85%, at least about 90%, at least about 95%, or about 100% of the cells remain viable after passing through each of the multiple constrictions. The viability of the cells can be measured using any suitable methods known in the art. In some aspects, the viability of the cells can be measured using a Nucleocounter NC-200, an Orflo Moxi Go II Cell Counter, or both.

Exemplary microfluidic channels containing cell-deforming constrictions for use in the methods disclosed herein are described in US Publ. No. 2020/0277566 A1, US Publ. No. 2020/0332243 A1, US Publ. No. 2020/0316604 A1, U.S. Provisional Appl. No. 63/131,423, and U.S. Provisional Appl. No. 63/131,430, each of which is incorporated herein by reference in its entirety.

In some aspects, a microfluidic channel described herein (i.e., comprising a constriction) includes a lumen and is configured such that a cell suspended in a buffer (e.g., cell suspension) can pass through the channel. Microfluidic channels useful for the present disclosure can be made using any suitable materials available in the art, including, but not limited to, silicon, metal (e.g., stainless steel), plastic (e.g., polystyrene), ceramics, glass, crystalline substrates, amorphous substrates, polymers (e.g., Poly-methyl methacrylate (PMMA), PDMS, Cyclic Olefin Copolymer (COC)), or combinations thereof. In some aspects, the material is silicon. Fabrication of the microfluidic channel can be performed by any method known in the art, including, but not limited to, dry etching for example deep reactive ion etching, wet etching, photolithography, injection molding, laser ablation, SU-8 masks, or combinations thereof. In some aspects, the fabrication is performed using dry etching.

In some aspects, a microfluidic channel useful for the present disclosure comprises an entrance portion, a center point, and an exit portion. In some aspects, the cross-section of one or more of the entrance portion, the center point, and/or the exit portion can vary. For example, the cross-section can be circular, elliptical, an elongated slit, square, hexagonal, or triangular in shape.

The entrance portion defines a constriction angle. In some aspects, by modulating (e.g., increasing or decreasing) the constriction angle, any clogging of the constriction can be reduced or prevented. In some aspects, the angle of the exit portion can also be modulated. For example, in some aspects, the angle of the exit portion can be configured to reduce the likelihood of turbulence that can result in non-laminar flow. In some aspects, the walls of the entrance portion and/or the exit portion are linear. In some aspects, the walls of the entrance portion and/or the exit portion are curved.

In some aspects, the length, depth, and/or width of the constriction can vary. In some aspects, by modulating (e.g., increasing or decreasing) the length, depth, and/or width of the constriction, the delivery efficiency of a payload can be regulated. As used herein, the term “delivery efficiency” refers to the amount of payload that is delivered into the cell. For instance, an increased delivery efficiency can occur when the total amount of payload that is delivered is increased.

In some aspects, the constriction has a length of less than about 1 μm. In some aspects the constriction has a length of about 0 μm to about 100 μm. In some aspects, the length of the constriction is less than about 0.1 μm, less than about 0.2 μm, less than about 0.3 μm, less than about 0.4 μm, less than about 0.5 μm, less than about 0.6 μm, less than about 0.7 μm, less than about 0.8 μm, less than about 0.9 μm, less than about 1 μm, less than about 2.5 μm, less than about 5 μm, less than about 7.5 μm, less than about 10 μm, less than about 12.5 μm, less than about 15 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2.5 μm, about 5 μm, about 7.5 μm, about 10 μm, about 12.5 μm, about 15 μm, about 20 μm, about 30 μm, about 40 μm, about 50 μm, about 60 μm, about 70 μm, about 80 μm, about 90 μm, or about 100 μm. In some aspects, the length of the constriction is about 10 μm. In some aspects, the constriction has a length of about 0 μm. For example, in some aspects, a microfluidic device (e.g., chip) useful for the present disclosure comprises a constriction that resembles two points of a diamond coming together, such that the length of the constriction is about 0 μm.

In some aspects, the width of the constriction is between about 0 μm to about 10 μm. In some aspects, the width of the constriction is less than about 0.1 μm, less than about 0.2 μm, less than about 0.3 μm, less than about 0.4 μm, less than about 0.5 μm, less than about 0.6 μm, less than about 0.7 μm, less than about 0.8 μm, less than about 0.9 μm, less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width of the constriction is about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about 8 μm, about 9 μm, or about 10 μm. In some aspects, width of the constriction is between about 3 μm to about 10 μm. In some aspects, the width of the constriction is about 6 μm.

In some aspects, the depth of the constriction is at least about 1 μm. In some aspects, the depth of the constriction is at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth of the constriction is about 5 μm, about 10 μm, about 20 μm, about 30 μm, about 40 μm, about 50 μm, about 60 μm, about 70 μm, about 80 μm, or about 90 μm. In some aspects, the depth of the constriction is about 70 μm.

In some aspects, the length of the constriction is about 10 μm, the width of the constriction is about 6 μm, and the depth of the constriction is about 70 μm. In some aspects, the length of the constriction is 10 μm, the width of the constriction is 6 μm, and the depth of the constriction is 70 μm.

In some aspects, the diameter of a constriction (e.g., contained within a microfluidic channel) is a function of the diameter of one or more cells that are passed through the constriction. Not to be bound by any one theory, in some aspects, the diameter of the constriction is less than that of the cells, such that a deforming force is applied to the cells as they pass through the constriction, resulting in the transient physical deformity of the cells.

Accordingly, in some aspects, the diameter of the constriction (also referred to herein as “constriction size”) is about 20% to about 99% of the diameter of the cell. In some aspects, the constriction size is about 20%, about 30%, about 40%, about 50%, about 60%, about 70%, about 80%, about 90%, or about 99% of the cell diameter. As is apparent from the present disclosure, by modulating (e.g., increasing or decreasing) the diameter of a constriction, the delivery efficiency of a payload into a cell can also be regulated.

### III.C.2. Surfaces Having Pores

In some aspects, a constriction described herein comprises a pore, which is contained in a surface. Non-limiting examples of pores contained in a surface that can be used with the present disclosure are described in, e.g., US Publ. No. 2019/0382796 A1, which is incorporated herein by reference in its entirety.

In some aspects, a surface useful for the present disclosure (i.e., comprising one or more pores that can cause a physical deformity in a cell as it passes through the pore) can be made using any suitable materials available in the art and/or take any one of a number of forms. Non- limiting examples of such materials include synthetic or natural polymers, polycarbonate, silicon, glass, metal, alloy, cellulose nitrate, silver, cellulose acetate, nylon, polyester, polyethersulfone, polyacrylonitrile (PAN), polypropylene, PVDF, polytetrafluorethylene, mixed cellulose ester, porcelain, ceramic, or combinations thereof.

In some aspects, the surface comprises a filter. In some aspects, the filter is a tangential flow filter. In some aspects, the surface comprises a membrane. In some aspects, the surface comprises a sponge or sponge-like matrix. In some aspects, the surface comprises a matrix. In some aspects, the surface comprises a tortuous path surface. In some aspects, the tortuous path surface comprises cellulose acetate.

The surface disclosed herein (i.e., comprising one or more pores) can have any suitable shape known in the art. Where the surface has a 2-dimensional shape, the surface can be, without limitation, circular, elliptical, round, square, star-shaped, triangular, polygonal, pentagonal, hexagonal, heptagonal, or octagonal. In some aspects, the surface is round in shape. Where the surface has a 3-dimensional shape, in some aspects, the surface can be, without limitation, cylindrical, conical, or cuboidal.

As is apparent from the present disclosure, a surface that is useful for the present disclosure (e.g., comprising one or more pores) can have various cross-sectional widths and thicknesses. In some aspects, the cross-sectional width of the surface is between about 1 mm and about 1 m. In some aspects, the surface has a defined thickness. In some aspects, the surface thickness is uniform. In some aspects, the surface thickness is variable. For example, in some aspects, certain portions of the surface are thicker or thinner than other portions of the surface. In such aspects, the thickness of the different portions of the surface can vary by about 1% to about 90%. In some aspects, the surface is between about 0.01 μm to about 5 mm in thickness.

The cross-sectional width of the pores can depend on the type of cell that is being targeted with a payload. In some aspects, the pore size is a function of the diameter of the cell of cluster of cells to be targeted. In some aspects, the pore size is such that a cell is perturbed (i.e., physically deformed) upon passing through the pore. In some aspects, the pore size is less than the diameter of the cell. In some aspects, the pore size is about 20% to about 99% of the diameter of the cell. In some aspects, the pore size is about 20%, about 30%, about 40%, about 50%, about 60%, about 70%, about 80%, about 90%, or about 99% of the diameter of the cell. In some aspects, the pore size is about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about μm, about 9 μm, about 10 μm, about 11 μm, about 12 μm, about 13 μm, about 14 μm, or about 15 μm or more.

The entrances and exits of a pore can have a variety of angles. In some aspects, by modulating (e.g., increasing or decreasing) the pore angle, any clogging of the pore can be reduced or prevented. In some aspects, the flow rate (i.e., the rate at which a cell or a suspension comprising the cell passes through the pore) is between about 0.001 mL/cm/sec to about 100 L/cm/sec. For example, the angle of the entrance or exit portion can be between about 0 and about 90 degrees. In some aspects, the pores have identical entrance and exit angles. In some aspects, the pores have different entrance and exit angles. In some aspects, the pore edge is smooth, e.g., rounded or curved. As used herein, a “smooth” pore edge has a continuous, flat, and even surface without bumps, ridges, or uneven parts. In some aspects, the pore edge is sharp. As used herein, a “sharp” pore edge has a thin edge that is pointed or at an acute angle. In some aspects, the pore passage is straight. As used herein, a “straight” pore passage does not contain curves, bends, angles, or other irregularities. In some aspects, the pore passage is curved. As used herein, a “curved” pore passage is bent or deviates from a straight line. In some aspects, the pore passage has multiple curves, e.g., about 2, about 3, about 4, about 5, about 6, about 7, about 8, about 9, about 10 or more curves.

The pores can have any shape known in the art, including a 2-dimensional or 3-dimensional shape. The pore shape (e.g., the cross-sectional shape) can be, without limitation, circular, elliptical, round, square, star-shaped, triangular, polygonal, pentagonal, hexagonal, heptagonal, and octagonal. In some aspects, the cross-section of the pore is round in shape. In some aspects, the 3-dimensional shape of the pore is cylindrical or conical. In some aspects, the pore has a fluted entrance and exit shape. In some aspects, the pore shape is homogenous (i.e., consistent or regular) among pores within a given surface. In some aspects, the pore shape is heterogeneous (i.e., mixed or varied) among pores within a given surface.

A surface useful for the present disclosure can have a single pore. In some aspects, a surface useful for the present disclosure comprises multiple pores. In some aspects, the pores encompass about 10% to about 80% of the total surface area of the surface. In some aspects, the surface contains about 1.0×10⁵ to about 1.0×10³⁰ total pores. In some aspects, the surface comprises between about 10 and about 1.0×10¹⁵ pores per mm² surface area.

The pores can be distributed in numerous ways within a given surface. In some aspects, the pores are distributed in parallel within a given surface. In some aspects, the pores are distributed side-by-side in the same direction and are the same distance apart within a given surface. In some aspects, the distribution of the pores is ordered or homogeneous. In such aspects, the pores can be distributed in a regular, systematic pattern, or can be the same distance apart within a given surface. In some aspects, the distribution of the pores is random or heterogeneous. For instance, in some aspects, the pores are distributed in an irregular, disordered pattern, or are different distances apart within a given surface.

In some aspects, multiple surfaces are used, such that a cell passes through multiple pores, wherein the pores are on different surfaces. In some aspects, multiple surfaces are distributed in series. The multiple surfaces can be homogeneous or heterogeneous in surface size, shape, and/or roughness. The multiple surfaces can further contain pores with homogeneous or heterogeneous pore size, shape, and/or number, thereby enabling the simultaneous delivery of a range of payloads into different cell types.

In some aspects, an individual pore, e.g., of a surface that can be used with the present disclosure, has a uniform width dimension (i.e., constant width along the length of the pore passage). In some aspects, an individual pore has a variable width (i.e., increasing or decreasing width along the length of the pore passage). In some aspects, pores within a given surface have the same individual pore depths. In some aspects, pores within a given surface have different individual pore depths. In some aspects, the pores are immediately adjacent to each other. In some aspects, the pores are separated from each other by a distance. In some aspects, the pores are separated from each other by a distance of about 0.001 μm to about 30 mm.

In some aspects, the surface is coated with a material. The material can be selected from any material known in the art, including, without limitation, Teflon, an adhesive coating, surfactants, proteins, adhesion molecules, antibodies, anticoagulants, factors that modulate cellular function, nucleic acids, lipids, carbohydrates, transmembrane proteins, or combinations thereof. In some aspects, the surface is coated with polyvinylpyrrolidone. In some aspects, the material is covalently attached to the surface. In some aspects, the material is non-covalently attached to the surface. In some aspects, the surface molecules are released at the cells pass through the pores.

In some aspects, the surface has modified chemical properties. In some aspects, the surface is hydrophilic. In some aspects, the surface is hydrophobic. In some aspects, the surface is charged. In some aspects, the surface is positively and/or negatively charged. In some aspects, the surface can be positively charged in some regions and negatively charged in other regions. In some aspects, the surface has an overall positive or overall negative charge. In some aspects, the surface can be any one of smooth, electropolished, rough, or plasma treated. In some aspects, the surface comprises a zwitterion or dipolar compound. In some aspects, the surface is plasma treated.

In some aspects, the surface is contained within a larger module. In some aspects, the surface is contained within a syringe, such as a plastic or glass syringe. In some aspects, the surface is contained within a plastic filter holder. In some aspects, the surface is contained within a pipette tip.

### III.D. Cell Perturbation

As described herein, as a cell passes through a constriction, it becomes physically deformed, such that there is a perturbation (e.g., a hole, tear, cavity, aperture, pore, break, gap, perforation) in the cell membrane of the cell. Such perturbation in the cell membrane is temporary and sufficient for any of the payloads (e.g., reprogramming factor) described herein to be delivered into the cell. Cells have self-repair mechanisms that allow the cells to repair any disruption in their cell membrane. See Blazek et al., Physiology (Bethesda) 30(6): 438-48 (November 2015), which is incorporated herein by reference in its entirety. Accordingly, in some aspects, once the cells have passed through the constriction (e.g., microfluidic channel or pores), the perturbations in the cell membrane can be reduced or eliminated, such that the payload that was delivered into the cell does not exit the cell.

In some aspects, the perturbation in the cell membrane lasts from about 1.0×10⁻⁹ seconds to about 2 hours after the pressure is removed (e.g., cells have passed through the constriction). In some aspects, the cell perturbation lasts for about 1.0×10⁻⁹ second to about 1 second, for about 1 second to about 1 minute, or for about 1 minute to about 1 hour. In some aspects, the cell perturbation lasts for between about 1.0×10⁻⁹ second to about 1.0×10⁻¹ second, between about 1.0×10⁻⁹ second to about 1.0×10⁻² second, between about 1.0×10⁻⁹ second to about 1.0×10⁻³ second, between about 1.0×10⁻⁹ second to about 1.0×10⁻⁴ second, between about 1.0×10⁻⁹ second to about 1.0×10⁻⁵ second, between about 1.0×10⁻⁹ second to about 1.0×10⁻⁶ second, between about 1.0×10⁻⁹ second to about 1.0×10⁻⁷ second, or between about 1.0×10⁻⁹ second to about 1.0×10⁻⁸ second. In some aspects, the cell perturbation lasts for about 1.0×10⁻⁸ second to about 1.0×10⁻¹ second, for about 1.0×10⁻⁷ second to about 1.0×10⁻¹ second, about 1.0×10⁻⁶ second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. The cell perturbations (e.g., pores or holes) created by the methods described herein are not formed as a result of assembly of polypeptide subunits to form a multimeric pore structure such as that created by complement or bacterial hemolysins.

In some aspects, as the cell passes through the constriction, the pressure applied to the cells temporarily imparts injury to the cell membrane that causes passive diffusion of material through the perturbation. In some aspects, the cell is only deformed or perturbed for a brief period of time, e.g., on the order of 100 μs or less to minimize the chance of activating apoptotic pathways through cell signaling mechanisms, although other durations are possible (e.g., ranging from nanoseconds to hours). In some aspects, the cell is deformed for less than about 1.0×10⁻⁹ second to less than about 2 hours. In some aspects, the cell is deformed for less than about 1.0×10⁻⁹ second to less than about 1 second, less than about 1 second to less than about 1 minute, or less than about 1 minute to less than about 1 hour. In some aspects, the cell is deformed for about 1.0×10⁻⁹ second to about 2 hours. In some aspects, the cell is deformed for about 1.0×10⁻⁹ second to about 1 second, about 1 second to about 1 minute, or about 1 minute to about 1 hour. In some aspects, the cell is deformed for between any one of about 1.0×10⁻⁹ second to about 1.0×10⁻¹ second, about 1.0×10⁻⁹ second to about 1.0×10⁻² second, about 1.0×10⁻⁹ second to about 1.0×10⁻³ second, about 1.0×10⁻⁹ second to about 1.0×10⁻⁴ second, about 1.0×10⁻⁹ second to about 1.0×10⁻⁵ second, about 1.0×10⁻⁹ second to about 1.0×10⁻⁶ second, about 1.0×10⁻⁹ second to about 1.0×10⁻⁷ second, or about 1.0×10⁻⁹ second to about 1.0×10⁻⁸ second. In some aspects, the cell is deformed or perturbed for about 1.0×10⁻⁸ second to about 1.0×10⁻¹ second, for about 1.0×10⁻⁷ second to about 1.0×10⁻¹ second, about 1.0×10⁻⁶ second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. In some aspects, deforming the cell includes deforming the cell for a time ranging from, without limitation, about 1 μs to at least about 750 μs, e.g., at least about 1 82 s, at least about 10 μs, at least about 50 μs, at least about 100 μs, at least about 500 μs, or at least about 750 μs.

In some aspects, the delivery of a saRNA encoding a payload (e.g., reprogramming factor) into the cell occurs simultaneously with the cell passing through the constriction. In some aspects, delivery of the saRNA encoding a payload into the cell can occur after the cell passes through the constriction (i.e., when perturbation of the cell membrane is still present and prior to cell membrane of the cells being restored). In some aspects, delivery of the saRNA encoding a payload into the cell occurs on the order of minutes after the cell passes through the constriction. In some aspects, a perturbation in the cell after it passes through the constriction is corrected within the order of about five minutes after the cell passes through the constriction.

In some aspects, the viability of a cell (e.g., stem cell or PBMC) after passing through a constriction is about 5% to about 100%. In some aspects, the cell viability after passing through the constriction is at least about 5%, at least about 10%, at least about 20%, at least about 30%, at least about 40%, at least about 50%, at least about 60%, at least about 70%, at least about 75%, at least about 80%, at least about 85%, at least about 90%, at least about 95%, or at least about 99%. In some aspects, the cell viability is measured from about 1.0×10⁻² second to at least about 10 days after the cell passes through the constriction. For example, the cell viability can be measured from about 1.0×10⁻² second to about 1 second, about 1 second to about 1 minute, about 1 minute to about 30 minutes, or about 30 minutes to about 2 hours after the cell passes through the constriction. In some aspects, the cell viability is measured about 1.0×10⁻² second to about 2 hours, about 1.0×10⁻² second to about 1 hour, about 1.0×10⁻² second to about 30 minutes, about 11.0×10⁻² second to about 1 minute, about 1.0×10⁻² second to about 30 seconds, about 1.0×10⁻² second to about 1 second, or about 1.0×10⁻² second to about 0.1 second after the cell passes through the constriction. In some aspects, the cell viability is measured about 1.5 hours to about 2 hours, about 1 hour to about 2 hours, about 30 minutes to about 2 hours, about 15 minutes to about 2 hours, about 1 minute to about 2 hours, about 30 seconds to about 2 hours, or about 1 second to about 2 hours after the cell passes through the constriction. In some aspects, the cell viability is measured about 2 hours to about 5 hours, about 5 hours to about 12 hours, about 12 hours to about 24 hours, or about 24 hours to about 10 days after the cell passes through the constriction.

### III.E. Delivery Parameters

As is apparent from the present disclosure, a number of parameters can influence the delivery efficiency of a saRNA described herein (e.g., encoding a reprogramming factor) into a cell using the squeeze processing methods provided herein. Accordingly, by modulating (e.g., increasing or decreasing) one or more of the delivery parameters, the delivery of a payload into a cell can be improved. Therefore, in some aspects, the present disclosure relates to a method of increasing the delivery of a payload (e.g., reprogramming factor) into a cell, wherein the method comprises modulating one or more parameters under which a cell suspension is passed through a constriction, wherein the cell suspension comprises a population of the cells, and wherein the one or more parameters increase the delivery of a payload into one or more cells of the population of cells compared to a reference parameter. As described elsewhere in the present disclosure, the payload can be in contact with the population of cells before, during, or after the squeezing step.

In some aspects, by modulating one or more of the delivery parameters, the delivery of the payload (e.g., reprogramming factor) into the one or more cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, compared to a delivery of the payload agent into a corresponding cell using the reference parameter.

In some aspects, the one or more delivery parameters that can be modulated to increase the delivery efficiency of a parameter comprises a cell density (i.e., the concentration of the cells present, e.g., in the cell suspension), pressure, or both. Additional examples of delivery parameters that can be modulated are provided elsewhere in the present disclosure.

In some aspects, the cell density is about 1×10⁷ cells/mL, about 2×10⁷ cells/mL, about 3×10⁷ cells/mL, about 4×10⁷ cells/mL, about 5×10⁷ cells/mL, about 6×10⁷ cells/mL, about 7×10⁷ cells/mL, about 8×10⁷ cells/mL, about 9×10⁷ cells/mL, about 1×10⁸ cells/mL, about 1.1×10⁸ cells/mL, about 1.2×10⁸ cells/mL, about 1.3×10⁸ cells/mL, about 1.4×10⁸ cells/mL, about 1.5×10⁸ cells/mL, about 2.0×10⁸ cells/mL, about 3.0×10⁸ cells/mL, about 4.0×10⁸ cells/mL, about 5.0×10⁸ cells/mL, about 6.0×10⁸ cells/mL, about 7.0×10⁸ cells/mL, about 8.0×10⁸ cells/mL, about 9.0×10⁸ cells/mL, or about 1.0×10⁹ cells/mL or more. In some aspects, the cell density is between about 6×10⁷ cells/mL and about 1.2×10⁸ cells/mL.

In some aspects, the pressure is about 20 psi, about 25 psi, about 30 psi, about 35 psi, about 40 psi, about 50 psi, about 55 psi, about 60 psi, about 65 psi, about 70 psi, about 75 psi, about 80 psi, about 85 psi, about 90 psi, about 95 psi, about 100 psi, about 110 psi, about 120 psi, about 130 psi, about 140 psi, about 150 psi, about 160 psi, about 170 psi, about 180 psi, about 190 psi, or about 200 psi or more. In some aspects, the pressure is between about 30 psi and about 90 psi.

In some aspects, the particular type of device (e.g., microfluidic chip) can also have an effect on the delivery efficiency of a payload described herein (e.g., reprogramming factor). In the case of a microfluidic chip, different chips can have different constriction parameters, e.g., length, depth, and width of the constriction; entrance angle, exit angle, length, depth, and width of the approach region, etc. As described herein, such variables can influence the delivery of a payload into a cell using the squeeze processing methods of the present disclosure. In some aspects, the length of the constriction is up to 100 μm. For instance, in some aspects, the length is about 1 μm, about 5 μm, 10 μm, about 20 μm, about 30 μm, about 40 μm, about 50 μm, about 60 μm, about 70 μm, about 80 μm, about 90 μm, or about 100 μm. In some aspects, the length of the constriction is less than 1 μm. In some aspects, the length of the constriction is less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the constriction has a length of about 10 μm.

In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width is between about 3 μm to about 10 μm. In some aspects, the width is about 3 μm, about 4 μm, about 5 μm, about 6 μm, about 7 μm, about 8 μm, about 9 μm, or about 10 μm. In some aspects, the width of the constriction is about 6 μm.

In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth is between about 5 μm to about 90 μm. In some aspects, the depth is about 5 μm, about 10 μm, about 15 μm, about 20 μm, about 30 μm, about 40 μm, about 50 μm, about 60 μm, about 70 μm, about 80 μm, or about 90 μm. In some aspects, the depth of the constriction is about 70 μm.

In some aspects, the length is about 10 μm, the width is about 6 μm, and depth is about 70 μm.

Additional examples of parameters that can influence the delivery of a payload into the cell include, but are not limited to, the dimensions of the constriction (e.g., length, width, and/or depth), the entrance angle of the constriction, the surface properties of the constrictions (e.g., roughness, chemical modification, hydrophilic, hydrophobic), the operating flow speeds, payload concentration, the amount of time that the cell recovers, or combinations thereof. Further parameters that can influence the delivery efficiency of a payload (e.g., reprogramming factor) can include the velocity of the cell in the constriction, the shear rate in the constriction, the viscosity of the cell suspension, the velocity component that is perpendicular to flow velocity, and time in the constriction. Such parameters can be designed to control delivery of the payload.

In some aspects, the temperature used in the methods of the present disclosure can also have an effect on the delivery efficiency of the payloads into the cell, as well as the viability of the cell. In some aspects, the squeeze processing method is performed between about −5° C. and about 45° C. For example, the methods can be carried out at room temperature (e.g., about 20° C.), physiological temperature (e.g., about 37° C.), higher than physiological temperature (e.g., greater than about 37°° C. to 45° C. or more), or reduced temperature (e.g., about −5° C. to about 4° C.), or temperatures between these exemplary temperatures.

Various methods can be utilized to drive the cells through the constrictions. For example, pressure can be applied by a pump on the entrance side (e.g., gas cylinder, or compressor), a vacuum can be applied by a vacuum pump on the exit side, capillary action can be applied through a tube, and/or the system can be gravity fed. Displacement based flow systems can also be used (e.g., syringe pump, peristaltic pump, manual syringe or pipette, pistons, etc.). In some aspects, the cells are passed through the constrictions by positive pressure. In some aspects, the cells are passed through the constrictions by constant pressure or variable pressure. In some aspects, pressure is applied using a syringe. In some aspects, pressure is applied using a pump. In some aspects, the pump is a peristaltic pump or a diaphragm pump. In some aspects, pressure is applied using a vacuum. In some aspects, the cells are passed through the constrictions by g-force. In some aspects, the cells are passed through the constrictions by capillary pressure.

In some aspects, fluid flow directs the cells through the constrictions. In some aspects, the fluid flow is turbulent flow prior to the cells passing through the constriction. Turbulent flow is a fluid flow in which the velocity at a given point varies erratically in magnitude and direction. In some aspects, the fluid flow through the constriction is laminar flow. Laminar flow involves uninterrupted flow in a fluid near a solid boundary in which the direction of flow at every point remains constant. In some aspects, the fluid flow is turbulent flow after the cells pass through the constriction. The velocity at which the cells pass through the constrictions can be varied. In some aspects, the cells pass through the constrictions at a uniform cell speed. In some aspects, the cells pass through the constrictions at a fluctuating cell speed.

In some aspects, a combination treatment is used to deliver a payload, e.g., the methods described herein followed by exposure to an electric field downstream of the constriction. In some aspects, the cell is passed through an electric field generated by at least one electrode after passing through the constriction. In some aspects, the electric field assists in delivery of a payload to a second location inside the cell such as the cell nucleus. In some aspects, one or more electrodes are in proximity to the cell-deforming constriction to generate an electric field. In some aspects, the electric field is between about 0.1 kV/m to about 100 MV/m. In some aspects, an integrated circuit is used to provide an electrical signal to drive the electrodes. In some aspects, the cells are exposed to the electric field for a pulse width of between about 1 ns to about 1 s and a period of between about 100 ns to about 10 s.

### III.F. Therapeutic Uses

In some aspects, the present disclosure relates to the use of the cells produced using the squeeze processing methods described herein to treat various diseases or disorders. As is apparent from the present disclosure, the methods and compositions provided herein can be useful for diseases and disorders where cell replacement therapies can be used as a treatment. By replacing cells that are damaged with the cells produced using the methods provided herein, in some aspects, one or more functions associated with the damaged cells can be restored, and thereby, treat the disease or disorder. For instance, in some aspects, neurons that are produced using the squeeze processing methods provided herein could be administered to a subject suffering from a neurological disorder. The administration of such neurons could be useful in improving one or more symptoms associated with the neurological disorder.

As used herein, the terms “neurological disorder” and “neuroimmunological disorder” can be used interchangeably and refer to diseases and disorders of either the central or peripheral nervous system. Unless specified otherwise, the term “neurological disorders” and “neuroimmunological disorders” comprises all diseases or disorders of the nervous system, including autoimmune disorders. Non-limiting examples of neurological disorders that can be treated with the present disclosure include a brain tumor, neoplastic meningitis, leptomeningeal cancer disease (LMD), amyotrophic lateral sclerosis (ALS), frontotemporal dementia (FTD), Parkinson's disease (PD), Huntington's disease (HD), Alzheimer's disease (AD), or combinations thereof. In some aspects, the neurological disorder is Parkinson's disease.

### IV. Compositions of the Disclosures

In some aspects, the disclosure provides a system for delivery of a payload (e.g., reprogramming factor) into a cell, the system comprising a microfluidic channel described herein, a cell suspension comprising a plurality of the cells and the payload; wherein the constriction is configured such that the plurality of cells can pass through the microfluidic channel, wherein the passing of the plurality of cells causes a deformity and disruption of the cell membrane of the cell, allowing the payload to enter the cell.

In some aspects, the disclosure provides a system for delivering a payload, the system comprising a surface with pores, a cell suspension comprising a plurality of the cells and the payload; wherein the surface with pores is configured such that the plurality of cells can pass through the pores, wherein the passing of the plurality of cells causes a deformity and disruption of the cell membrane of the cell, allowing the payload to enter the cell. In some aspects, the surface is a filter or a membrane. In some aspects of the above aspects, the system further comprises at least one electrode to generate an electric field. In some aspects, the system is used to deliver a payload into a cell by any of the methods described herein. The system can include any aspect described for the methods disclosed above, including microfluidic channels or a surface having pores to provide cell-deforming constrictions, cell suspensions, cell perturbations, delivery parameters. In some aspects, the delivery parameters, such as operating flow speeds, cell and compound concentration, velocity of the cell in the constriction, and the composition of the cell suspension (e.g., osmolarity, salt concentration, serum content, cell concentration, pH, etc.) are optimized for delivery of a payload (e.g., reprogramming factor) into the cell.

In some aspects, the disclosure provides a cell produced using any of the methods provided herein (e.g., neuron). In some aspects, provided herein is a cell comprising a perturbation in the cell membrane, wherein the perturbation is due to one or more parameters which deform the cell (e.g., delivery parameters described herein), thereby creating the perturbation in the cell membrane of the cell such that a payload (e.g., reprogramming factor) can enter the cell. In some aspects, provided herein is a cell comprising a payload (e.g., reprogramming factor), wherein the payload entered the cell through a perturbation in the cell membrane, which was due to one or more parameters which deform the cell (e.g., delivery parameters described herein) and thereby creating the perturbation in the cell membrane of the cell such that the payload entered the cell. In some aspects, such cells can comprise any of the cells described herein (e.g., stem cells or PBMCs).

In some aspects, the present disclosure provides a composition comprising a plurality of cells, wherein the plurality of cells were produced by any of the methods provided herein. Also provided herein is a composition comprising a population of cells and a payload (e.g., reprogramming factor) under one or more parameters, which result in deformation of one or more cells of the population of cells and thereby creating perturbations in the cell membrane of the one or more cells, and wherein the perturbations in the cell membrane allows the payload to enter the one or more cells.

Also provided are kits or articles of manufacture for use in delivering into a cell a payload (e.g., reprogramming factor) as described herein. In some aspects, the kits comprise the compositions described herein (e.g. a microfluidic channel or surface containing pores, cell suspensions, and/or payload) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture can further be sterilized and/or sealed.

The present disclosure also provides kits comprising components of the methods described herein and can further comprise instruction(s) for performing said methods to deliver a payload (e.g., reprogramming factor) into a cell. The kits described herein can further include other materials, including other buffers, diluents, filters, needles, syringes, and package inserts with instructions for performing any methods described herein; e.g., instructions for delivering a payload into a cell.

The following examples are offered by way of illustration and not by way of limitation.

### EXAMPLES

### Example 1: Analysis of the GFP-Puro saRNA Expression and Duration in iPSCs

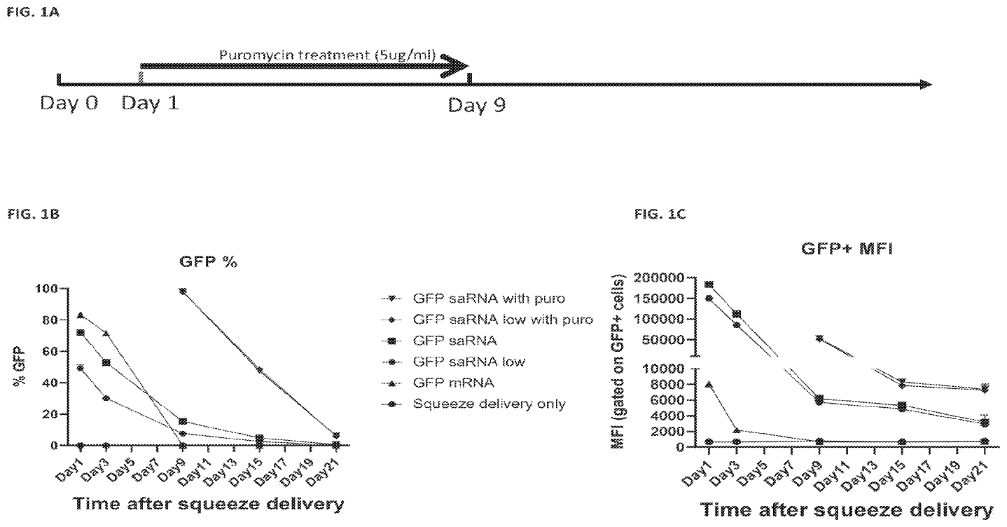

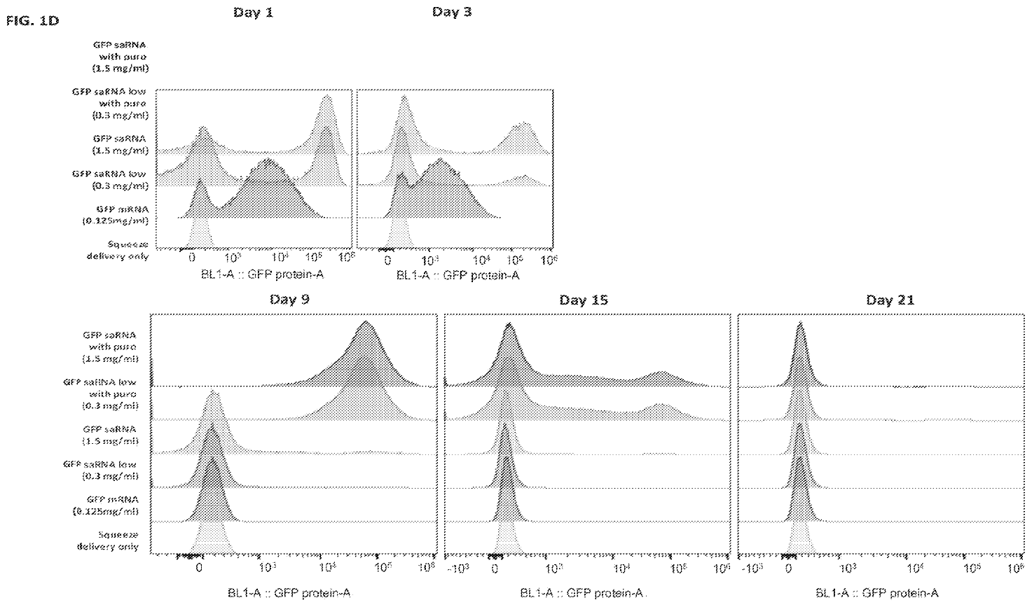

To determine the GFP expression and duration of GFP-Puro saRNA, iPSCs were treated with Accutase and dissociated into single cells. iPSCs were prepared at a density of 1×10⁸ cells/mL. The prepared iPSCs were then combined with GFP-Puro saRNA (1.5 mg/ml or 0.3 mg/ml). The cell suspension was added to a constriction channel with the following dimensions at room temperature in mTeSR™ Plus Basal Medium: length of 10 μm, width of 6 μm, and depth of 70 μm. Cells squeeze processed with GFP mRNA (non-self-amplifying) (0.125 mg/ml) were used as positive control and cells squeeze processed without any cargo was used as negative control. GFP-Puro saRNA and GFP mRNA concentration was adjusted based on its size to have equal molecular concentration. Following squeeze-processing, the squeeze-loaded iPSCs were transferred to mTeSR™ Plus Basal Medium with supplement and incubated at 37° C. Subset of GFP-Puro saRNA delivered iPSCs were treated with puromycin at 5 ug/ml from day 1 to day9. (See FIG. 1A). The cells were collected for analysis of green fluorescent signal by flow cytometry at day 1, 3, 9, 15, and 21.

As shown in FIGS. 1B-1D, GFP mRNA and GFP saRNA had similar squeeze delivery efficiency. However, GFP MFI was over 20-fold higher for saRNA vs mRNA at day1. Additionally, puromycin selection was highly effective (95%+GFP positive) and extended GFP expression beyond 9 days of culture. After puromycin withdrawal, GFP % dropped from 95% (at day9) to 6% (at day21), suggesting that without puromycin selection, the cells lost GFP-Puro saRNA. The ratio of GFP+saRNA cells vs non-delivered cells decreased from day1-3 while the MFI of the GFP+mRNA cells remained the same, suggesting that there was a proliferation disadvantage in the saRNA-delivered iPSCs. The MFI of GFP positive cells remained high in GFP-Puro saRNA at day21 and was comparable to GFP mRNA at day1.

Overall, these results demonstrate that GFP saRNA expressed for a higher and longer period of time compared to GFP mRNA.

### Example 2: Analysis of Ascl1 saRNA-Derived Cells after Squeeze Processing

To assess whether the delivery of Ascl1 saRNA to cells using squeeze processing methods described herein can induce dopamine neuron differentiation, iPSCs were treated with Accutase and dissociated into single cells. Then, the iPSCs were prepared at a density of 1×10⁸ cells/mL and combined in a cell suspension with one of the following conditions: (1) GFP saRNA; (2) Ascl1 saRNA; (3) Ascl1 saRNA+5TFs (FoxA2, Lmx1a, NR4A2, Pitx3, and EN1) mRNA (non-self-amplifying). The cell suspensions were added to constrictions with the same dimensions as that described in Example 1 at room temperature with a pressure of 60 psi. After the cells had passed through the constriction, the cells were collected and transferred to StemFlex basal medium with supplement and incubated for 6 hours at 37° C. At 6 hours, the cells were washed with PBS to remove unwanted debris. A mixture of 1:1 ratio of StemFlex basal medium with N2B medium+B27 (100×) with 5 ug/ml puromycin was added. The mixture was incubated for 18 hours. The medium was then removed, and a mixture of NBM medium+B27 (50×)+BDNF+GDNF (1:1000) was added. Total RNA was collected from the cells at day1 and day4, and RT-qPCR was used to analyze the expression of general neuronal markers (NeuroD1) and dopamine lineage markers (FoxA2, Pitx3, Lmx1a, NR4A2, and TH).

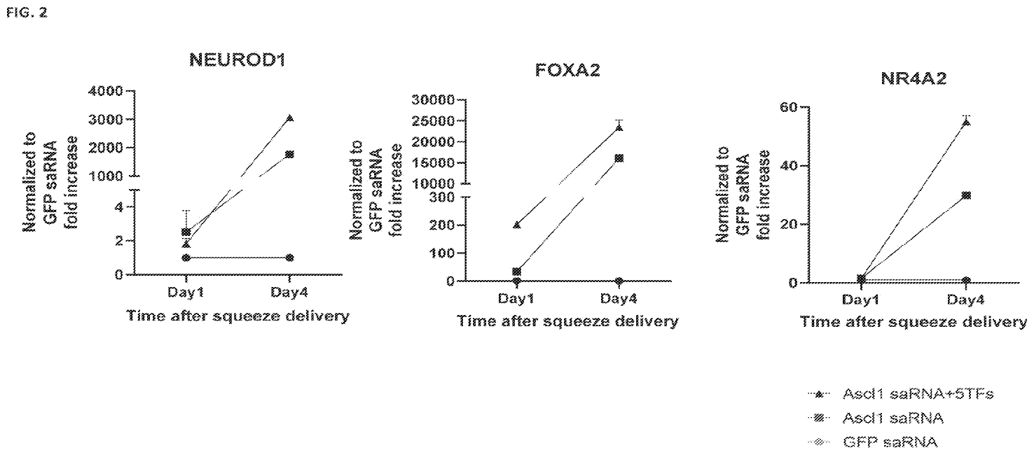

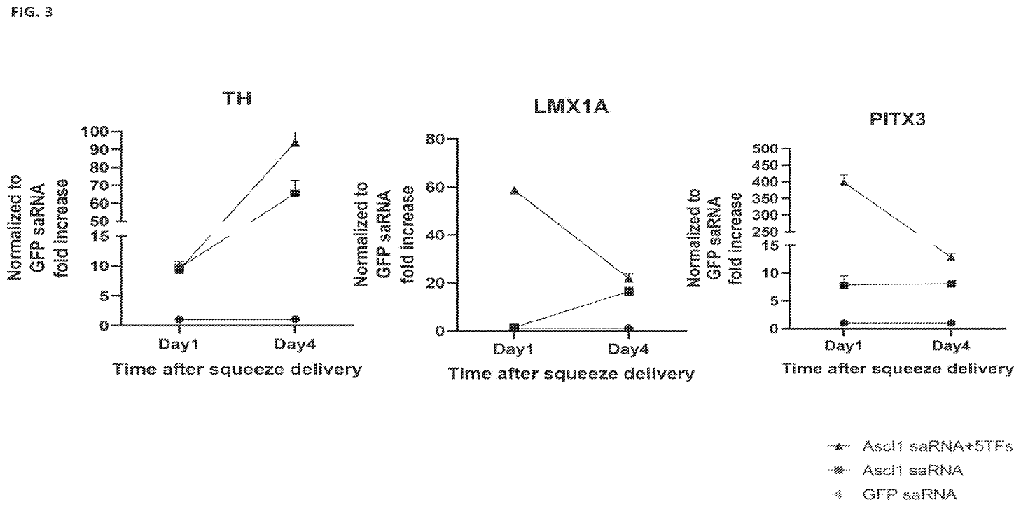

As shown in FIGS. 2 and 3, all the markers were upregulated as compared to GFP saRNA. Most markers had higher expression at day4 than day 1. All the dopamine lineage markers tested were higher in the codelivery of Ascl1 saRNA with 5TFs (FoxA2, Lmx1a, NR4A2, Pitx3, and EN1) mRNA compared to Ascl1 saRNA alone, suggesting the additional effects from 5TFs mRNA.

### Example 3: Analysis of Ascl1 saRNA-Derived Dopamine Neurons after Squeeze Processing

To assess whether the delivery of Ascl1 saRNA to cells using squeeze processing methods described herein can generate dopamine neurons, iPSCs were treated with Accutase and dissociated into single cells. Then, the iPSCs were prepared at a density of 1×10⁸ cells/mL and combined in a cell suspension with Ascl1 saRNA+5TFs (FoxA2, Lmx1a, NR4A2, Pitx3, and EN1) mRNA (non-self amplifying). The cell suspensions were added to constrictions with the same dimensions as that described in Example 1 at room temperature with a pressure of 60 psi. After the cells had passed through the constriction, the cells were collected and transferred to StemFlex basal medium with supplement and incubated for 6 hours at 37° C. At 6 hours, the cells were washed with PBS to remove unwanted debris. A mixture of 1:1 ratio of StemFlex basal medium with N2B medium+B27 (100×) with 5 ug/ml puromycin was added. The mixture was incubated for 18 hours. The medium was then removed, and a mixture of NBM medium+B27(50×)+BDNF and GDNF (1:1000) was added. Half medium removal every 2 days with NBM medium+B27 (50×) +BDNF and GDNF (1:1000). Cells were fixed at day14, and immunofluorescence stained for dopamine neuronal marker tyrosine hydroxylase TH, early neuronal marker TUJ1, and mature neuronal marker MAP2.

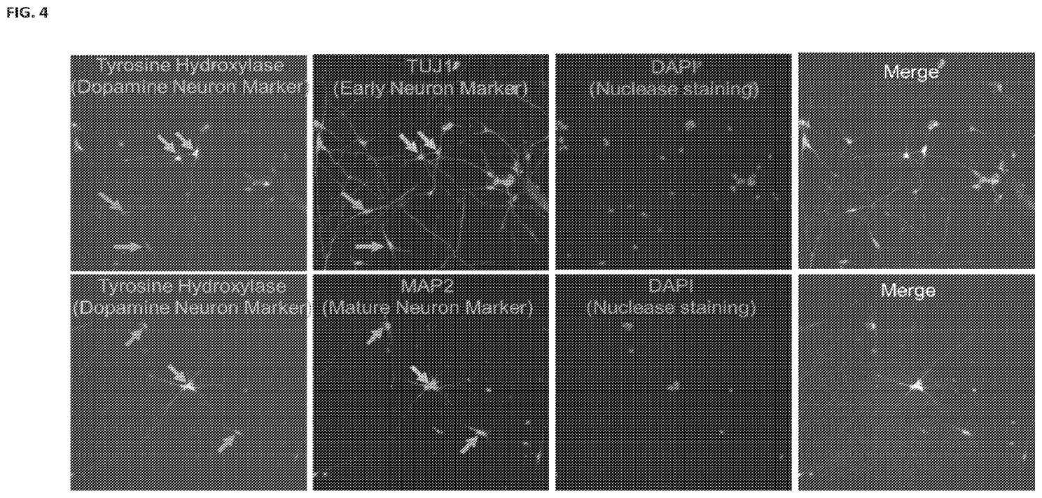

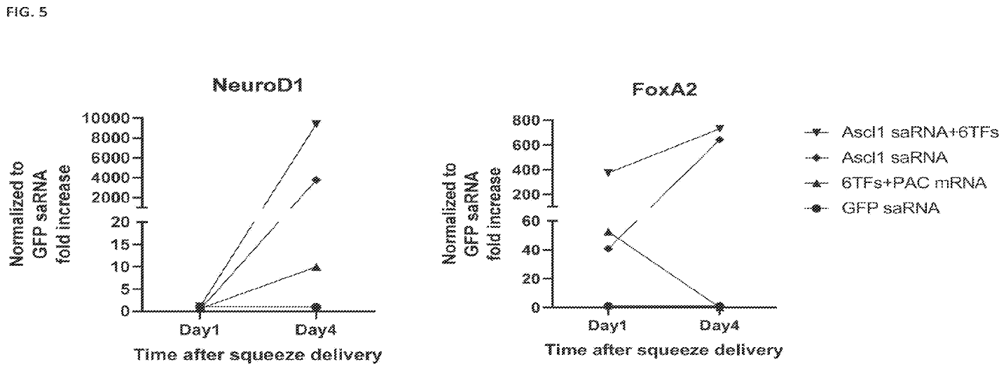

As shown in FIG. 4, all neuron-like cells expressed early neuronal marker TUJ1. Some cells had high expression of dopamine neuronal marker tyrosine hydroxylase (green arrows) while some cells had lower expression (red arrows). All neuron-like cells expressed mature neuronal marker MAP2 while some cells also had high expression of dopamine neuronal marker tyrosine hydroxylase TH (green arrows) and some cells had low expression of TH (red arrows). These results further confirmed the earlier data and demonstrated that the delivery of saRNAs using squeeze processing can effective induce the differentiation of iPSCs into neurons.

### Example 4: Analysis of Kinetic Difference between mRNA (Non-Self-Amplifying) and saRNA after Squeeze Processing

To assess the kinetic difference between mRNA and saRNA, iPSCs were treated with Accutase and dissociated into single cells. Then, the iPSCs were prepared at a density of 1×10⁸ cells/mL and combined in a cell suspension with one of the following conditions: (1) GFP saRNA; (2) 6TFs (Ascl1, FoxA2, Lmx1a, NR4A2, Pitx3, and EN1)+PAC mRNA (non-self amplifying) (3) Ascl1 saRNA; (4) Ascl1 saRNA+6TFs (Ascl1, FoxA2, Lmx1a, NR4A2, Pitx3, and EN1) mRNA (non-self amplifying). The cell suspensions were added to constrictions with the same dimensions as that described in Example 1 at room temperature with a pressure of 60 psi. After the cells had passed through the constriction, the cells were collected and transferred to StemFlex basal medium with supplement and incubated for 6 hours at 37°° C. At 6 hours, the cells were washed with PBS to remove unwanted debris. A mixture of 1:1 ratio of StemFlex basal medium with N2B medium+B27 (100×) with 5 ug/ml puromycin was added. The mixture was incubated for 18 hours. The medium was then removed, and a mixture of NBM medium+B27 (50×)+BDNF+GDNF (1:1000) was. Total RNA was collected from the cells at day 1 and day4, and RT-qPCR was used to analyze the expression of general neuronal markers (NeuroD1) and dopamine lineage markers (FoxA2, Pitx3, Lmx1a, NR4A2, and TH).

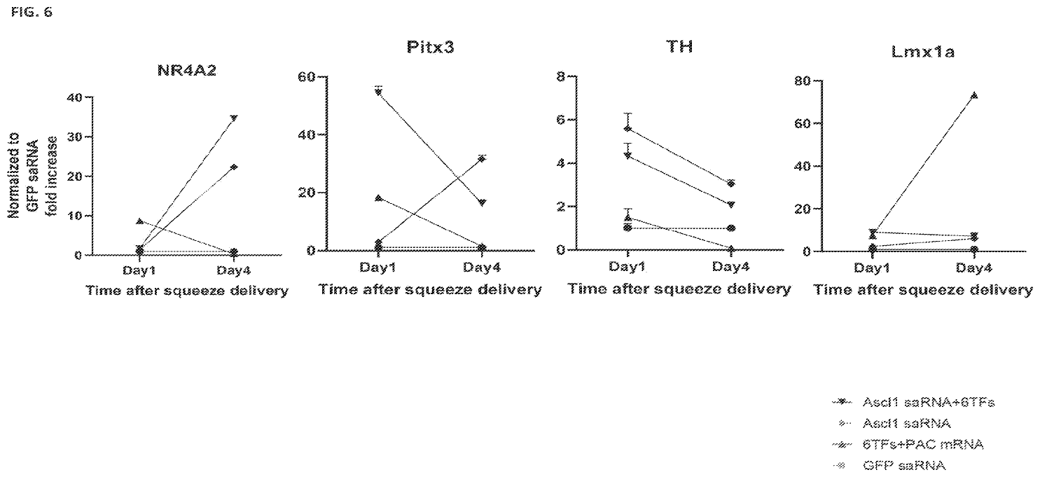

As shown in FIGS. 5 and 6, most markers showed that 6TFs mRNA had higher target gene expression at day 1 compared to day4 indicating transient nature of mRNA. For samples with saRNA, most target genes had an increase from day 1 to day 4, showing impact of longer-term expression from saRNA.

Collectively, the above results demonstrate that saRNAs encoding reprogramming factors, particularly when delivered using squeeze processing methods, allow for long-term expression of the encoded reprogramming factors and effective differentiation of iPSCs into neurons.

### Incorporation by Reference

All publications, patents, patent applications and other documents cited in this application are hereby incorporated by reference in their entireties for all purposes to the same extent as if each individual publication, patent, patent application or other document were individually indicated to be incorporated by reference for all purposes.

### Equivalents

While various specific aspects have been illustrated and described, the above specification is not restrictive. It will be appreciated that various changes can be made without departing from the spirit and scope of the disclosure(s). Many variations will become apparent to those skilled in the art upon review of this specification.

1-9. (canceled)

10. A method of reprogramming a cell, comprising passing a cell suspension, which comprises the cell, through a constriction under a set of parameters, wherein passing the cell suspension through the constriction under the set of parameters causes a perturbation within the membrane of the cell, such that a self-amplifying RNA encoding a reprogramming factor can enter the cell through the perturbation when contacted with the cell, and thereby, reprogramming the cell.

11-12. (canceled)

13. A method of enhancing the reprogramming of a cell, comprising passing a cell suspension, which comprises the cell, through a constriction under a set of parameters, wherein passing the cell suspension through the constriction under the set of parameters causes a perturbation within the membrane of the cell, such that a self-amplifying RNA encoding a reprogramming factor can enter the cell through the perturbation when contacted with the cell, and thereby, resulting in enhanced reprogramming of the cell, as compared to a reference method,
wherein the reference method comprises passing the cell suspension through the constriction under the set of parameters and contacting the cell suspension with a non-self-amplifying RNA encoding the reprogramming factor, or wherein the reference method does not comprise passing the cell suspension through the constriction under the set of parameters, and wherein the cell suspension is contacted with the self-amplifying RNA.

14-15. (canceled)

16. The method of claim 13, wherein the enhanced reprogramming of the cell comprises (i) a greater number of cells having been reprogrammed, (ii) a shorter duration of time required to reprogram cells, or (iii) both (i) and (ii).

17-18. (canceled)

19. The method of claim 10, wherein the reprogramming of the cell comprises inducing the cell to differentiate into a neuron.

20. The method of claim 10, wherein the reprogramming of the cell comprises inducing the expression of other reprogramming factor in the cell.

21-30. (canceled)

31. The method of claim 10, wherein the reprogramming factor comprises a transcription factor.

32. The method of claim 31, wherein the reprogramming factor comprises a neurogenin-2 (Ngn2; Neurog2), Atonal BHLH Transcription Factor 1 (Atoh1), Achaete-Scute Family BHLH Transcription Factor 1 (Ascl1), Nuclear receptor subfamily 4 group A member 2 (NR4A2), LIM Homeobox Transcription Factor 1 Alpha (Lmx1a), Engrailed homobox 1 (EN1), POU Class 3 Homeobox 2 (POU3F2; Brn2), Myelin Transcription Factor 1 Like (Myt11), Forkhead Box A2 (Foxa2), Paired Like Homeodomain 3 (Pitx3), SRY-Box Transcription Factor 2 (SOX2), micro-RNA 124 (mir124), or combinations thereof.

33. The method of claim 10, which further comprises contacting the cell suspension with a payload, such that the payload can also enter the cell through the perturbation when contacted with the cell, wherein the self-amplifying RNA and the payload are contacted with the cell concurrently or sequentially.

34-41. (canceled)

42. The method of claim 33, wherein the payload comprises:
(i) a nucleic acid, a polypeptide, a lipid, a carbohydrate, a small molecule, a metal-containing compound, an antibody, a transcription factor, a nanoparticle, a liposome, a fluorescently tagged molecule, or combinations thereof; or
(ii) an additional reprogramming factor.

43-48. (canceled)

49. The method of claim 10, wherein the cell comprises stem cells, somatic cells, or both.

50. The method of claim 49, wherein:
(i) the stem cells are induced pluripotent stem cells (iPSCs), embryonic stem cells, tissue-specific stem cells, mesenchymal stem cells, or combinations thereof; and/or
(ii) the somatic cells comprise blood cells.

51-55. (canceled)

56. The method of claim 10, wherein the set of parameters is selected from a cell density; pressure; length, width, and/or depth of the constriction; diameter of the constriction; diameter of the cells; temperature; entrance angle of the constriction; exit angle of the constriction; length, width, and/or width of an approach region; surface property of the constriction; operating flow speed; payload concentration; viscosity, osmolarity, salt concentration, serum content, and/or pH of the cell suspension; time in the constriction; shear rate in the constriction; type of payload, or combinations thereof.

57-76. (canceled)

77. The method of claim 33, comprising contacting the cell suspension with multiple self-amplifying RNAs, multiple payloads, or both (i) prior to the passing of the cell suspension through the constriction, (ii) during the passing of the cell suspension through the constriction, (iii) after the passing of the cell suspension through the constriction, or (iv) any combination of (i) to (iii).

78-91. (canceled)

92. The method of claim 10, further comprising passing the cell suspension through a plurality of constrictions, wherein:
(i) each constriction of the plurality of constrictions is the same;
(ii) one or more of the constrictions of the plurality of constrictions are different;
(iii) each constriction of the plurality of constrictions is associated with the same self-amplifying RNA; or
(iv) one or more of the plurality of constrictions is associated with a different self-amplifying RNA.

93-100. (canceled)

101. The method of claim 10, further comprising contacting the cell suspension with an additional compound, wherein the additional compound is a nucleic acid encoding an enzyme that confers resistance to an antibiotic.

102-121. (canceled)

122. A cell comprising a reprogramming factor, wherein the reprogramming factor entered the cell through a perturbation in the cell membrane due to a set of parameters which deformed the cell, thereby causing the perturbation in the cell membrane of the cell, and allowing a self-amplifying RNA encoding the reprogramming factor to enter the cell through the perturbation.

123-127. (canceled)

128. The method of claim 13, wherein the reprogramming of the cell comprises inducing the cell to differentiate into a neuron.

129. The method of claim 13, wherein the reprogramming of the cell comprises inducing the expression of other reprogramming factor in the cell.

130. The method of claim 13, wherein the reprogramming factor comprises a transcription factor.

131. The method of claim 130, wherein the reprogramming factor comprises a neurogenin-2 (Ngn2; Neurog2), Atonal BHLH Transcription Factor 1 (Atoh1), Achaete-Scute Family BHLH Transcription Factor 1 (Ascl1), Nuclear receptor subfamily 4 group A member 2 (NR4A2), LIM Homeobox Transcription Factor 1 Alpha (Lmx1a), Engrailed homobox 1 (EN1), POU Class 3 Homeobox 2 (POU3F2; Brn2), Myelin Transcription Factor 1 Like (Myt11), Forkhead Box A2 (Foxa2), Paired Like Homeodomain 3 (Pitx3), SRY-Box Transcription Factor 2 (SOX2), micro-RNA 124 (mir124), or combinations thereof.
